(19) United States
(12) Patent Application Publication
(10) Pub. No.: US 2026/0137738 A1
(43) Pub. Date: May 21, 2026

(54) Novel Salmonella Enterica-Specific Bacteriophage OPT-SAL01, and Antibacterial Composition Comprising Same

(71) Applicant: Optipharm Co., Ltd., Cheongju-Si, Chungcheongbuk-do (KR)

(72) Inventors: Young Ju Kim, Sejong (KR); Ryeon Hoe Seo, Cheongju-si, Chungcheongbuk-do (KR); Hyunil Kim, Seoul (KR)

(21) Appl. No.: 18/878,777

(22) PCT Filed: Jul. 1, 2022
(86) PCT No.: PCT/KR2022/009513

§ 371 Date: Oct. 31, 2025

(30) Foreign Application Priority Data
Jun. 30, 2022 (KR) 10-2022-0080277

(51) Int. Cl. A61K 35/76; A01N 63/40; A01P 1/00; A23K 10/16; A61P 31/04; C12N 7/00

(52) U.S. Cl. CPC A61K 35/76; A01N 63/40; A01P 1/00; A23K 10/16; A61P 31/04; C12N 7/00; C12N 2795/00021; C12N 2795/00031; C12N 2795/00032

(57) ABSTRACT

The present invention relates to a novel bacteriophage OPT-SALO1 with specific killing ability for Salmonella enterica, an antibiotic composition comprising the bacteriophage, a composition for adding to a feed, a feed, a disinfectant or a cleaning agent, and a method for preventing or treating infectious diseases caused by Salmonella enterica comprising a step of administering the bacteriophage to a subject.

### TECHNICAL FIELD

The present disclosure relates to a novel bacteriophage OPT-SAL01 with a specific bactericidal activity against Salmonella enterica, an antibiotic composition, a composition for adding to a feed, a feed, a disinfectant or a cleaning agent including the bacteriophage, and a method for preventing or treating infectious diseases caused by Salmonella enterica including administering the bacteriophage to a subject.

### BACKGROUND ART

Salmonella is a Gram-negative, facultative anaerobic, non-spore-forming, rod-shaped bacterium belonging to the family Enterobacteriaceae, most strains of which are motile due to the presence of flagella. Salmonella is broadly classified into two species, Salmonella enterica and Salmonella bongori, and is a pathogenic microorganism that causes food poisoning in humans and various types of Salmonellosis in livestock such as chickens, pigs, and cattle. Among them, Salmonella enterica has more than 2,600 serotypes that have been currently reported based on serological classification. The serotypes are divided into typhoid and non-typhoid types, and non-typhoid bacteria include serotypes of Salmonella agona, Salmonella albany, Salmonella typhimurium as a causative bacterium of rat typhus, Salmonella enteritidis as an Enteritidis bacterium, Salmonella gallinarum as a causative bacterium of fowl typhus, and Salmonella pullorum as a causative bacterium of Pullorum, all of which have zoonotic infectivity capable of causing gastrointestinal diseases and infecting not only humans but also other animals.

Salmonella albany is a non-typhoid bacterium and a major pathogen that causes food poisoning in humans and Salmonellosis in livestock such as chickens, pigs, and cattle to cause acute or chronic enteritis and sepsis, as well as pneumonia, arthritis, miscarriage, and other conditions. Salmonella albany also has the characteristics of being able to survive for long periods of time even in harsh and dry conditions and survive even in the air. Due to these characteristics, Salmonella mostly spreads through the fecal and oral routes in livestock, but may also spread through the air to be easily contagious. Salmonella infects humans through livestock, and can contaminate human foods through direct and indirect contamination opportunities throughout the entire process of livestock farming, slaughtering, and processing. Mass-produced protein feed for animals and poultry can cause widespread infections in these animals. For example, in the UK, Salmonella infections that lasted for several years in the late 1960s and early 1970s, caused by fishmeal imported from Peru and fed to poultry, resulted in a significant number of human cases. In 2017, in France, an increase in infected patients was reported in among infants who consumed formula milk contaminated with Salmonella.

Currently, antibiotics are mainly used to treat salmonellosis, but there is a difficulty in that the infected bacteria need to be identified, and antibiotic prescriptions must be applied based on the identification of bacteria. In addition, when Salmonella infects animals, Salmonella often penetrates cells, proliferates, and spreads, making it difficult for antibiotics, drugs, and other probiotics to penetrate and act. Therefore, the best method to deal with salmonellosis is prevention. Among various preventive methods, antibiotics were used as growth promoters in compound feed for industrial animals, but recently, due to a problem of antibiotic resistance, antibiotics have begun to be prohibited from being used as growth promoters in compound feed, and in order to reduce misuse during treatment, antibiotics cannot be used sufficiently. As the use of antibiotics decreases, the incidence of bacterial diseases increases, thereby increasing the need for methods to control bacterial diseases without using antibiotics. One of these studies is a method using bacteriophages, which specifically and effectively kill bacteria existing in nature.

Meanwhile, a bacteriophage refers to a bacteria-specific virus that infects specific bacteria and inhibits and suppresses the growth of the infected bacteria. The bacteriophages have the ability to proliferate inside bacterial cells after infection, and kill bacteria by destroying the cell walls of host bacteria when progeny bacteriophages burst out of the bacteria after proliferation. A bacterial infection method of bacteriophages is very highly specific, so that the types of bacteriophages that may infect specific bacteria are partially limited. In other words, specific bacteriophages may infect only specific categories of bacteria, and as a result, the specific bacteriophages kill only specific bacteria and do not affect other bacteria. Therefore, the use of bacteriophages has recently attracted significant attention as a means of dealing with bacterial diseases. Interest in bacteriophages may be higher than ever due to the growing preference for nature-friendly methods. With the growing potential for development as an alternative to conventional antibiotics, bacteriophages are once again gaining attention as anti-bacterial agents.

### DISCLOSURE

### Technical Problem

Accordingly, the present inventors completed the present disclosure by isolating a bacteriophage with specific lytic activity against Salmonella enterica and analyzing the morphological and genetic characteristics of the isolated bacteriophage to confirm that the bacteriophage may selectively kill Salmonella, particularly Salmonella albany.

Therefore, an object of the present disclosure is to provide a bacteriophage OPT-SAL01 (Accession Number: KCCM13132P) with a specific bactericidal activity against Salmonella enterica, consisting of a base sequence as set forth in SEQ ID NO: 1; and a composition including the same.

Another object of the present disclosure is to provide a method for preventing or treating infectious diseases caused by Salmonella enterica, including administering a bacteriophage OPT-SAL01 to a subject in need thereof.

Yet another object of the present disclosure is to provide a method for inhibiting Salmonella enterica, including contacting a bacteriophage OPT-SAL01 with a food, a feed or a container.

### Technical Solution

In order to achieve the object, an aspect of the present disclosure provides a bacteriophage OPT-SAL01 (Accession Number: KCCM13132P) with a specific bactericidal activity against Salmonella enterica, consisting of a base sequence as set forth in SEQ ID NO: 1.

Another aspect of the present disclosure provides an antibiotic composition including a bacteriophage OPT-SAL01.

Yet another aspect of the present disclosure provides a composition for adding to a feed including a bacteriophage OPT-SAL01 and a feed including the same.

Still another aspect of the present disclosure provides a disinfectant including a bacteriophage OPT-SAL01.

Still another aspect of the present disclosure provides a cleaning agent including a bacteriophage OPT-SAL01.

Still another aspect of the present disclosure provides a method for preventing or treating infectious diseases caused by Salmonella enterica, including administering a bacteriophage OPT-SAL01 to a subject in need thereof.

Still another aspect of the present disclosure provides a method for inhibiting Salmonella enterica, including contacting a bacteriophage OPT-SAL01 with a food, a feed or a container.

### Advantageous Effects

According to the present disclosure, the bacteriophage OPT-SALO1 has very high specificity for Salmonella enterica compared to conventional chemical substances such as antibiotics, and has advantages of excellent lytic activity and resistance to physicochemical stimuli by infecting Salmonella enterica and proliferating within the bacteria. In addition, since the bacteriophage OPT-SAL01 of the present disclosure does not infect hosts other than bacteria, such as humans, animals, and plants, the bacteriophage has an advantage of being able to solve the problem of antibiotic-resistant bacteria due to the misuse of antibiotics, the problem of antibiotic residues in foods, and the problem of a wide host range. Therefore, the bacteriophage OPT-SAL01 of the present disclosure can be used in various fields such as prevention or treatment of infectious diseases caused by Salmonella enterica, antibiotic compositions, feed additives, feed, disinfectants, or cleaning agents.

### DESCRIPTION OF DRAWINGS

FIG. 1 is a diagram showing a genetic map of the bacteriophage OPT-SAL01 according to the present disclosure.

FIG. 2 is a diagram showing the results of observing the morphological characteristics of the bacteriophage OPT-SAL01 using an electron microscope.

FIG. 3 is a diagram showing the results of evaluating the bactericidal activity of the bacteriophage OPT-SAL01.

FIG. 4 is a diagram showing the results of evaluating the pH stability of the bacteriophage OPT-SAL01.

FIG. 5 is a diagram showing the results of evaluating the thermal stability of the bacteriophage OPT-SAL01.

### BEST MODE

Hereinafter, the present disclosure will be described in detail.

The present disclosure provides a bacteriophage OPT-SAL01 with a specific bactericidal activity against for Salmonella enterica, consisting of a base sequence as set forth in SEQ ID NO: 1.

In the present disclosure, the term “bacteriophage” refers to a bacteria-specific virus that infects particular bacteria and inhibits or suppresses their growth, and includes viruses containing either single-stranded or double-stranded DNA or RNA as genetic material.

In a specific embodiment of the present disclosure, the Salmonella enterica may be at least one selected from the group consisting of Salmonella albany, Salmonella agona, Salmonella falkensee, Salmonella hardar, Salmonella indiana, Salmonella infantis, Salmonella kedougou, Salmonella kentucky, Salmonella molade, Salmonella montevideo, Salmonella orion, Salmonella paratyphi A, Salmonella rissen, Salmonella stanley, Salmonella tennessee, and Salmonella virchow.

The bacteriophage OPT-SAL01 of the present disclosure has excellent lytic activity for various serotypes of Salmonella enterica. The bacteriophage OPT-SALO1 has the size of 190 to 210 nm, and belongs to the Myoviridae family, which has an icosahedral head and a contractile tail.

In a specific embodiment of the present disclosure, the bacteriophage OPT-SAL01 has excellent stability to heat and pH. More specifically, it is preferable that the bacteriophage OPT-SAL01 is stable at pH 4 to 11. In addition, it is preferable that the bacteriophage OPT-SAL01 is stable at 40 to 70° C.

The bacteriophage OPT-SAL01 has the whole genome size of 86,400 base pairs (bp), a GC content of 38.9%, and 120 open reading frames (ORFs). In addition, the complete nucleotide sequence of the bacteriophage OPT-SALO1 may be set forth as SEQ ID NO: 1.

In addition, the bacteriophage OPT-SAL01 may include a base sequence represented by SEQ ID NO: 1 as all or part of the whole genome. In addition, the bacteriophage OPT-SAL01 of the present disclosure may consist of a base sequence as set forth in SEQ ID NO: 1 and a functional equivalent of the base sequence. The functional equivalent means a sequence that has at least 70%, preferably 80% or more, more preferably 90% or more, and even more preferably 95% or more sequence homology with the base sequence as set forth in SEQ ID NO: 1, resulting from modification or substitution of the base sequence, and exhibiting substantially the same physiological activity as the base sequence as set forth in SEQ ID NO: 1.

The specific lytic activity against Salmonella enterica, acid resistance, alkali resistance and heat resistance enable the application of the bacteriophage OPT-SAL01 of the present disclosure to various temperatures and pH ranges by incorporating the bacteriophage OPT-SAL01 into compositions for preventing and treating infectious diseases caused by Salmonella enterica, as well as into various products including the bacteriophage OPT-SAL01 as an active ingredient.

The bacteriophage OPT-SAL01 of the present disclosure is a bacteriophage isolated from a poultry farm sample, which was named bacteriophage OPT-SAL01 by the present inventors and deposited on Feb. 18, 2022 according to the Budapest Treaty, at the Korean Culture Center of Microorganisms (Seoul, Korea) located at Hongjenae-2ga-gil, Seodaemun-gu, Seoul, Korea, and was assigned the accession number KCCM13132P.

Further, the present disclosure provides an antibiotic composition comprising the bacteriophage OPT-SAL01.

In the present disclosure, the term “antibiotic composition” means a preparation that is provided to animals in the form of a drug to kill bacteria, and is a generic term for preservatives, bactericides, antibiotics and antibacterial agents.

Since the bacteriophage OPT-SAL01 of the present disclosure has very high specificity for Salmonella enterica compared to conventional antibiotics, the bacteriophage OPT-SAL01 does not kill beneficial bacteria, but may kill only specific pathogens, and does not induce drug tolerance or resistance, making it suitable for use as a novel antibiotic with a longer lifecycle than conventional antibiotics.

Further, the present disclosure provides a composition for adding to a feed including the bacteriophage OPT-SAL01.

Antibiotics added to feeds used in livestock and fisheries have been used for the prevention of diseases, but the administration of antibiotics for prevention is problematic in that it increases the possibility of developing resistant bacteria, and antibiotic residues in livestock may be transmitted to humans. If antibiotics are absorbed into the human body through meat, they may cause antibiotic resistance and lead to the spread of diseases. In addition, since many types of antibiotics are mixed and fed with the feed, there is a problem of increasing the probability of multi-drug-resistant bacteria. As a new antibiotic for adding to feed that is more eco-friendly and also solves problems caused by the use of existing antibiotics, the bacteriophage OPT-SAL01 of the present disclosure may be used.

In addition, the present disclosure may provide a feed including the composition for adding to the feed, and the feed of the present disclosure may be manufactured by separately preparing a bacteriophage in the form of a feed additive and mixing the bacteriophage into the feed, or by directly adding the bacteriophage during the manufacture of the feed. The bacteriophage in the feed of the present disclosure may be in a liquid or dried form, preferably in a dried powder form. The drying method includes ventilation drying, natural drying, spray drying, and freeze drying, but is not limited thereto. The bacteriophage of the present disclosure may be mixed in powdered form at a composition ratio of 0.05 to 10 wt %, preferably 0.1 to 2 wt % of the feed weight. In addition, the feed may further include conventional additives capable of increasing the preservability of the feed in addition to the bacteriophage of the present disclosure.

Other non-pathogenic microorganisms may be further added to the composition for adding to the feed of the present disclosure. The microorganisms to be added may be selected from the group consisting of Bacillus species, such as Bacillus subtilis, capable of producing proteolytic enzymes, lipolytic enzymes and sugar converting enzymes, Lactobacillus strains (Lactobacillus sp.) with physiological activity and organic material decomposition ability under anaerobic conditions such as in the stomach of cattle, filamentous fungi such as Aspergillus oryzae, which have the effect of increasing the body weight of livestock, increasing milk production, and increasing the digestion-absorption rate of the feed, and yeasts such as Saccharomyces cerevisiae.

The feed including the bacteriophage OPT-SAL01 of the present disclosure includes vegetable-derived products such as grains, root fruits, food processing by-products, algae, fibers, pharmaceutical by-products, oils and fats, starches, meal, grain by-products, etc., and animal-derived products such as proteins, inorganic materials, fats and oils, minerals, single-celled proteins, zooplankton, leftover food, etc., but is not limited thereto.

The composition for adding to the feed of the present disclosure may include a binder, an emulsifier, a preservative, etc. that are added to prevent quality deterioration, and may include amino acids, vitamins, enzymes, probiotics, flavoring agents, non-protein nitrogen compounds, silicates, buffers, coloring agents, extractants, oligosaccharides, etc. that are added to the feed to increase efficacy, and may further include feed mixtures, etc.

Further, the present disclosure provides a drinking water additive including a bacteriophage OPT-SAL01.

The drinking water additive of the present disclosure may be used by separately manufacturing the bacteriophage OPT-SAL01 or a composition including the bacteriophage OPT-SAL01 in the form of a drinking water additive, which to be mixed with a feed or drinking water or directly added when manufacturing drinking water. As described above, the drinking water additive is mixed with drinking water and supplied to have the effect of continuously reducing the number of Salmonella.

In the present disclosure, the drinking water is not particularly limited, and any drinking water commonly used in the art may be used.

Further, the present disclosure provides a disinfectant including the bacteriophage OPT-SAL01.

The disinfectant including the bacteriophage OPT-SAL01 of the present disclosure having the specific bactericidal activity against Salmonella, for example, Salmonella enterica may be usefully used as hospital and health disinfectants to prevent hospital infection and may be used for general life disinfectants, disinfectants for food and cooking places and facilities, disinfection of various growing products such as buildings such as poultry farm and cattle shed, stock body, drinking water, straw litter, egg seats, transport vehicles, and tableware, and the like.

Further, the present disclosure provides a cleaning agent including the bacteriophage OPT-SAL01.

Since the bacteriophage OPT-SAL01 of the present disclosure has a specific bactericidal activity against Salmonella enterica, the bacteriophage OPT-SAL01 may also be used for washing the skin surface or body parts of livestock that have been exposed or are likely to be exposed to Salmonella.

Further, the present disclosure provides a pharmaceutical composition for preventing or treating infectious diseases caused by Salmonella enterica, including the bacteriophage OPT-SAL01.

Since the bacteriophage OPT-SAL01 of the present disclosure has a specific bactericidal activity against Salmonella enterica, the bacteriophage OPT-SAL01 may be used in a pharmaceutical composition for preventing or treating infectious diseases caused by Salmonella enterica.

In the present disclosure, the infectious disease caused by Salmonella enterica is a concept that collectively refers to infectious diseases that occur epidemically or acutely, and includes food poisoning, enteritis, pneumonia, gastroenteritis, asymptomatic infection, enteric fever, sepsis, and the like, in addition to salmonellosis, but is not limited thereto.

The salmonellosis is a general term for symptoms such as fever, headache, diarrhea, vomiting, etc. caused by Salmonella infection. The salmonellosis is broadly classified into a sepsis type with symptoms such as typhoid and an acute gastroenteritis type with food poisoning symptoms, and includes enteritis, food poisoning, acute bacteremia, and the like.

The pharmaceutical composition of the present disclosure includes 1×10³ to 1×10¹⁰ PFU/mL of bacteriophage, preferably 1×10⁶ to 1×10⁹ PFU/mL of bacteriophage. The term plaque forming unit (PFU) used herein is a unit that quantifies the formation of plaques by the bacteriophage.

The term ‘prevention’ in the present disclosure refers to any act of inhibiting disease or delaying the onset of disease through the administration of the composition.

The term “treatment” of the present disclosure refers to any action that improves the symptoms of the disease or inhibits or alleviates the disease and changes beneficially the disease by the administration of the composition.

The pharmaceutical composition of the present disclosure may further include a pharmaceutically acceptable carrier.

In the present disclosure, the term “pharmaceutically acceptable carrier” may refer to a carrier or a diluent that does not inhibit the biological activity and properties of a compound to be administered without stimulating organisms. In the composition formulated with a liquid solution, the pharmaceutically acceptable carrier is suitable for sterilization and living bodies and may use saline, sterilized water, Ringer's solution, buffered saline, albumin injection solution, dextrose solution, maltodextrin solution, glycerol, ethanol, and mixtures of at least one of these ingredients, and if necessary, may add other general additives such as antioxidants, buffers, bacteriostatic agents, and the like. In addition, the pharmaceutical composition may be formulated in injectable forms such as an aqueous solution, a suspension, and an emulsion, pills, capsules, granules, or tablets by further adding a diluent, a dispersant, a surfactant, a binder, and a lubricant.

The pharmaceutical composition of the present disclosure may be used by parenteral administration, nasal spray, and application or spraying to a diseased area, in addition to oral administration, and in the case of parenteral administration, the pharmaceutical composition may also be administered by intravenous administration, intraperitoneal administration, intramuscular administration, subcutaneous administration, or local administration.

Formulations for oral administration including the pharmaceutical composition of the present disclosure as an active ingredient may be prepared as, for example, tablets, troches, lozenges, aqueous or oily suspensions, prepared powders or granules, emulsions, hard or soft capsules, syrups or elixirs. For preparation into formulations such as tablets and capsules, the pharmaceutical composition may include a binder such as lactose, saccharose, sorbitol, mannitol, starch, amylopectin, cellulose or gelatin, an excipient such as dicalcium phosphate, a disintegrant such as corn starch or sweet potato starch, a lubricant such as magnesium stearate, calcium stearate, sodium stearyl fumarate or polyethylene glycol wax. In the case of capsule formation, the pharmaceutical composition may further contain a liquid carrier such as fatty oil, in addition to the aforementioned materials.

The formulations for parenteral administration including the pharmaceutical composition of the present disclosure as an active ingredient may be prepared in the form of an injection such as a subcutaneous injection, intravenous injection or intramuscular injection, a suppository injection, or a spray such as an aerosol that can be inhaled through the respiratory tract. For the preparation of the injectable formulation, the composition of the present disclosure may be mixed in water with a stabilizer or buffer to be prepared as a solution or suspension, and may be prepared for unit dosage of ampoules or vials. When formulated for the spray use such as aerosols, a propellant or the like may be mixed with additives to disperse a water-dispersed concentrate or wet powder.

Suitable application, spraying and dose of the pharmaceutical composition of the present disclosure vary depending on factors, such as a formulation method, an administration method, age, weight, and sex of the target animal and patient, the severity of disease symptoms, food, an administration time, an administration route, an excretion rate, and response sensitivity, and an ordinarily skilled physician or veterinarian may easily determine and prescribe a dose effective for desired treatment.

Further, the present disclosure provides a method for preventing or treating infectious diseases caused by Salmonella enterica, including administering the bacteriophage OPT-SAL01 to a subject in need thereof.

The method of the present disclosure is performed by administering the bacteriophage OPT-SAL01 to a subject suffering from or having a risk of developing infectious diseases caused by Salmonella enterica, such as epidemic or acute infectious diseases, including food poisoning, enteritis, pneumonia, gastroenteritis, asymptomatic infection, enteric fever, and sepsis, in addition to salmonellosis.

In the present disclosure, the term “subject” means all subjects capable of developing infectious diseases caused by Salmonella enterica, and includes mammals including humans, livestock, or poultry, but is not limited thereto.

The term “livestock” is a concept that refers to useful animals that have been domesticated and improved by humans and live together with humans, and includes, for example, pigs, cattle, chickens, horses, ducks or dogs, but is not limited thereto.

The term “poultry” is a general term for animals belonging to birds among livestock, and includes, for example, chickens, ducks, pheasants, quails, ostriches, geese, or turkeys, but is not limited thereto.

In the method of the present disclosure, the bacteriophage OPT-SAL01 or the composition may be administered to animals in the form of a pharmaceutical preparation, or may be administered by mixing it with the feed or drinking water of livestock and having the animals ingest the mixture.

In the method of the present disclosure, the bacteriophage OPT-SAL01 or the composition may be administered through various oral or parenteral routes, as long as being reach a target tissue, and specifically, may be administered by conventional methods, such as orally, rectally, topically, intravenously, intraperitoneally, intramuscularly, intraarterially, transdermally, intranasally, or through inhalation.

It is obvious to those skilled in the art that a total daily amount of the bacteriophage OPT-SAL01 suitable for administration in the method of the present disclosure may be determined by a doctor of treatment within the scope of sound medical judgment. However, it is desirable to apply a specific therapeutically effective amount for a specific subject differently, depending on various factors including the type and degree of response to be achieved, the age, body weight, general health condition, sex, and diet of a patient, an administration time, an administration route, a release rate of the composition, a duration of treatment, other drugs used in combination or simultaneously with a specific composition, and similar factors well known in the medical field.

According to yet another aspect of the present disclosure, the present disclosure provides a method for inhibiting Salmonella enterica, including contacting a bacteriophage OPT-SAL01 with a food, a feed or a container.

The bacteriophage OPT-SAL01 according to the present disclosure has excellent bactericidal activity against Salmonella enterica. Accordingly, the bacteriophage OPT-SAL01 of the present disclosure may kill Salmonella enterica that already exists by coming into contact with something in which Salmonella enterica is expected to exist, such as food, feed, or containers. In addition, the bacteriophage OPT-SAL01 of the present disclosure comes into contact with food, feed, or containers to prevent contamination with Salmonella enterica.

Redundant content is omitted in consideration of the complexity of the present specification, and terms not otherwise defined in the present specification shall have the meanings commonly used in the art to which the present disclosure pertains.

### [Modes]

Hereinafter, the present disclosure will be described in more detail through Examples. These Examples are merely illustrative of the present disclosure, and it will be apparent to those skilled in the art that the scope of the present disclosure should not be interpreted as being limited to these Examples.

### EXAMPLE 1. ISOLATION AND CULTURE OF NOVEL BACTERIOPHAGE

### 1.1 Screening and Isolation of Bacteriophages

Target bacteria, Salmonella enterica subspecies albany, that was bacteria that our company has isolated from a poultry farm and held, was used. To isolate bacteriophages, samples collected from sewage treatment plants in the Chungcheongbuk-do region were cultured overnight with shaking at 35° C. together with the target bacteria. The culture solution was centrifuged at 3,000 rpm for 20 minutes, the supernatant was filtered using a filter with a pore size of 0.45 μm, and the filtrate obtained above was used to isolate a bacteriophage specific to Salmonella albany through plaque assay. To screen bacteriophages, specifically, 150 ML of the pre-cultured target bacteria were placed in a sterilized tube, added with 3 mL of LB top agar [LB medium (10 g/L of tryptone, 5 g/L of yeast extract, 10 g/L of NaCl)+0.6% agar], mixed well, and then layered on a pre-prepared 2% LB agar plate. After left at room temperature for 30 minutes, 10 μL of the prepared sample filtrate was dropped onto plate, left at room temperature for 30 minutes, and then cultured at 35° C. for 24 hours to detect the presence or absence of bacteriophages. A soft agar overlay method was used to isolate pure phages from the samples in which the presence of bacteriophages had been confirmed. The samples in which the presence of bacteriophages was confirmed were appropriately diluted with saline solution, 100 μL of the sample dilution collected from each dilution step was mixed with 150 μL of the target bacteria culture solution, added with 3 mL of 0.6% LB top agar, mixed well, and then layered on a pre-prepared 2% LB agar plate, left, and cultured at 35° C. for 24 hours. Plaques formed on each cultured plate were identified and selected and suspended in 400 μL of an SM buffer (5.8 g/L of NaCl; 2 g/L of MgSO₄ 7H₂O₂; 50 mL of 1 M Tris-HCl (pH 7.5)) and left at room temperature for 4 hours. 100 μL of the bacteriophage solution was mixed with 500 μL of 0.6% LB top agar and bacterial culture solution, dispensed and layered onto a LB agar plate with 150 mm diameter, and cultured at 35° C. until complete lysis occurred. After the culture was completed, 15 mL of the SM buffer was added to the LB agar plate and the bacteriophage solution was recovered while stirring slowly at room temperature for 4 hours, and then centrifuged at 3,000 rpm for 20 minutes to collect only the supernatant and the supernatant was filtered through a 0.2 μm filter to recover the final bacteriophage solution.

### 1.2 Scale-Up Culture and Purification of Bacteriophages

In order to scale-up culture Salmonella albany-specific bacteriophages confirmed in Example 1.1 above, the final bacteriophage culture solution recovered in Example 1.1 was diluted to 1.0×10⁷ PFU/mL, and then mixed with 1.0×10⁹ CFU/mL of pre-cultured Salmonella albany to be a multiplicity of infection (MOI) of 0.01, and cultured at 35° C. and 100 rpm for 6 hours. After the culture was completed, the entire culture solution was centrifuged at 6,000 rpm for 30 minutes to obtain the supernatant, which was then filtered through a 0.2 μm filter to recover the final bacteriophage culture solution.

The isolated bacteriophage was named “bacteriophage OPT-SAL01” and deposited at the Korean Culture Center of Microorganisms (Seoul, Korea) on Feb. 18, 2022, and was assigned the accession number KCCM13132P.

### EXAMPLE 2. WHOLE GENOME ANALYSIS OF BACTERIOPHAGE OPT-SAL01

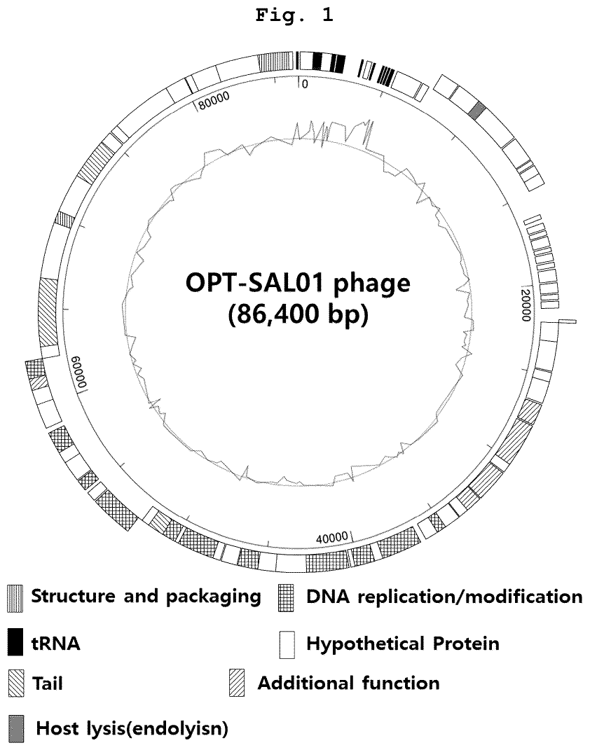

To analyze the whole genome of the bacteriophage OPT-SAL01, a bacteriophage OPT-SAL01 culture solution was added to 20 mM EDTA, 50 μg/mL of proteinase K, and 0.5% (w/v) SDS, and then incubated at 50° C. for 1 hour. The mixture was combined with an equal amount of phenol-chloroform-isopropyl alcohol (25:24:1), and then centrifuged at 12,000 rpm for 10 minutes at room temperature to recover the supernatant. The recovered supernatant was mixed with an equal volume of phenol-chloroform-isopropyl alcohol (25:24:1), centrifuged at 12,000 rpm for 10 minutes at room temperature to recover the supernatant, and then 3 M sodium acetate was added to make up 10% (v/v) of the total volume, followed by mixing. Here, the mixture was added with a double the volume of cold 95% ethanol, mixed, and left at −20° C. for 1 hour. After the reaction was completed, a precipitate was obtained by centrifugation at 12,000 rpm for 10 minutes at 0° C., washed twice with cold 70% ethanol, and then the ethanol was completely removed to recover a DNA pellet. The recovered pellet was dissolved in TE buffer (Tris-EDTA, pH 8.0) to a final volume of 50 μL, and the DNA concentration was then measured. For whole genome analysis, gene sequencing was performed using PacBio's Sequel (Macrogen, Korea). The genetic map of the bacteriophage OPT-SAL01 obtained from the sequencing result is shown in FIG. 1.

As shown in FIG. 1, it was confirmed that the bacteriophage OPT-SAL01 had the whole genome size of 86,400 bp, the GC content of 38.9%, and 120 open reading frames (ORFs). The whole gene sequence of the analyzed bacteriophage OPT-SAL01 is shown in SEQ ID NO: 1.

### EXAMPLE 3. MORPHOLOGICAL CHARACTERISTICS OF BACTERIOPHAGE OPT-SAL01

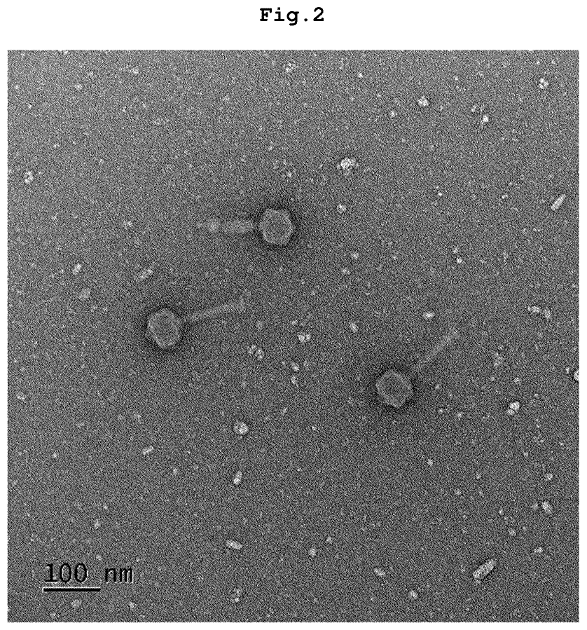

To determine the morphological characteristics of the bacteriophage OPT-SAL01, the culture solution was diluted in a 0.01% gelatin solution and fixed with a 2.5% glutaraldehyde solution. The solution was dropped on a carbon-coated mica plate (ca. 2.5×2.5 mm), and then adapted for 10 minutes and washed with sterile distilled water. The plate was mounted on copper grids, stained with 2% uranyl acetate for 30 to 60 seconds, dried, and then observed under a transmission electron microscope (Tecnai G2 Spirit Twin, Bio-Transmission electron microscope, 120 kV, 120,000 to 400,000). The result is shown in FIG. 2.

As shown in FIG. 2, the bacteriophage OPT-SAL01 was identified to belong to the Myoviridae family, having a size of 190 nm to 210 nm, an icosahedral head, and a contractile tail.

### EXAMPLE 4. INVESTIGATION OF INFECTIVITY AND BACTERICIDAL ACTIVITY OF BACTERIOPHAGE OPT-SAL01 FOR SALMONELLA

In order to determine whether the bacteriophage OPT-SAL01 exhibited lytic activity gainst Salmonella and various bacteria, the infectivity was determined through spot assay using strains isolated and held by Optipharm Co., Ltd. and strains provided by the KCTC and the like, and the results are shown in Tables 1 to 3.

TABLE 1

| | The number | The number of bacteria | |
|---|---|---|---|
| Name of target | of target | with confirmed lytic | Susceptibility |
| bacteria | bacteria | activity | ratio |
| Salmonella sp. | 114 | 112 | 98% |

TABLE 2

| Species | ID | OPT-SAL01 phage infection |
|---|---|---|
| Salmonella Albany | Wild type | Susceptible |
| Salmonella Agona | Wild type | Susceptible |
| Salmonella Falkensee | Wild type | Susceptible |
| Salmonella Hardar | Wild type | Susceptible |
| Salmonella Indiana | Wild type | Susceptible |
| Salmonella Infantis | Wild type | Susceptible |
| Salmonella Kedougou | Wild type | Susceptible |
| Salmonella Kentucky | Wild type | Susceptible |
| Salmonella Molade | Wild type | Susceptible |
| Salmonella Montevideo | Wild type | Susceptible |
| Salmonella Orion | Wild type | Susceptible |
| Salmonella Paratyphi A | Wild type | Susceptible |
| Salmonella Rissen | Wild type | Susceptible |
| Salmonella Stanley | Wild type | Susceptible |
| Salmonella Tennessee | Wild type | Susceptible |
| Salmonella Virchow | Wild type | Susceptible |

TABLE 3

| | | |
|---|---|---|
| Species | ID | OPT-SAL01 phage infection |

Gram negative bacteria

| | | |
|---|---|---|
| Acinetobacter baumannii | ATCC 19606 | Non-susceptibile |
| Citrobacter freundii | Wild type | Non-susceptibile |
| Escherichia coli | KCTC 1039 | Non-susceptibile |
| Klebsiella pneumoniae | Wild type | Non-susceptibile |
| Proteus miraibilis | KCTC 2566 | Non-susceptibile |
| Pseudomonas aeruginosa | KCTC2004 | Non-susceptibile |
| Serratia spp. | Wild type | Non-susceptibile |
| Yersinia enterocolitica | KCCM 41657 | Non-susceptibile |

As shown in Table 1, the bacteriophage OPT-SAL01 showed the lytic activity for 98% (112 strains) of 114 Salmonella strains to have excellent lytic activity for Salmonella.

As shown in Table 2, as a result of performing a susceptibility test on strains, with confirmed serotypes of Salmonella, it was shown that the bacteriophage OPT-SAL01 exhibited infectivity to various serotypes of Salmonella, indicating a wide range of lytic activity. In addition, when a spot assay was performed to confirm whether the bacteriophage OPT-SAL01 infected Gram-negative bacteria other than Salmonella, it was confirmed that the bacteriophage OPT-SAL01 was a Salmonella-specific bacteriophage because it did not infect any of the Gram-negative bacteria related to the intestinal tract used in the experiment.

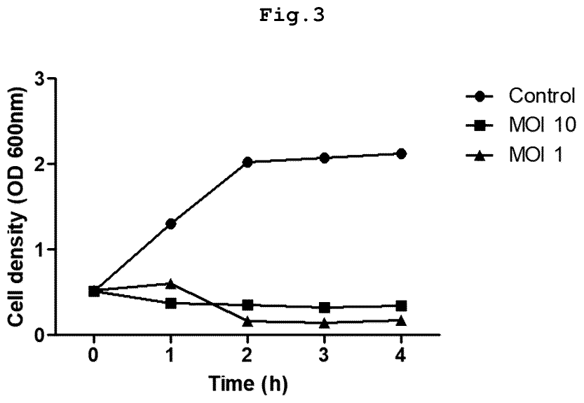

In addition, the bactericidal activity of the bacteriophage OPT-SAL01 against Salmonella albany was measured. Specifically, 10 mL of the bacterial culture solution was prepared so that the absorbance at 600 nm was approximately 0.5. The bacteriophage OPT-SAL01 was added to the bacterial culture solution to achieve MOIs of 1 and 10, respectively. At this time, the experiment was conducted using an experimental group inoculated only with bacteria as a control group, and the culture was performed at 35° C. for 4 hours, with the absorbance at 600 nm measured at 1-hour intervals. The results of the analysis of the bactericidal activity of bacteriophage OPT-SAL01 against Salmonella Albany are shown in FIG. 3.

As shown in FIG. 3, in the case of the control group without the addition of the bacteriophage OPT-SAL01, the absorbance at 600 nm was 2.12 after 4 hours, confirming that bacteria had grown. On the other hand, in the experimental group added with the bacteriophage OPT-SAL01, the absorbance at 600 nm after 4 hours decreased to 0.34 and 0.17 for MOI 10 and MOI 1, respectively, compared to the initial absorbance, indicating that the bacteria were effectively killed. The results indicate that the bacteriophage OPT-SAL01 has excellent bactericidal activity against Salmonella albany.

### EXAMPLE 5. INVESTIGATION OF PH STABILITY OF BACTERIOPHAGE OPT-SAL01

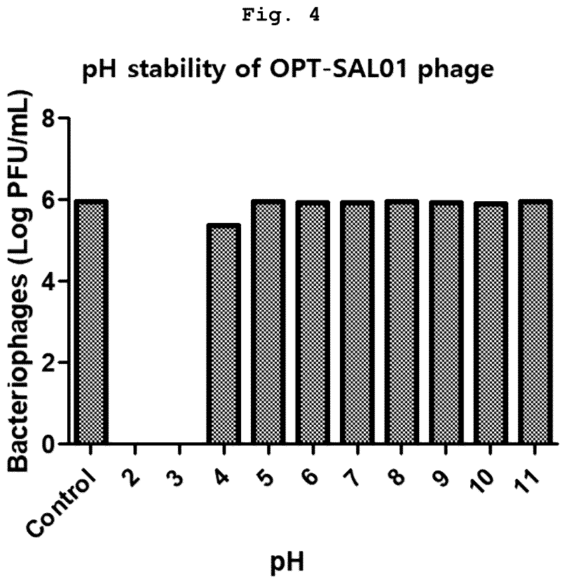

To confirm the pH stability of the bacteriophage OPT-SAL01, the number of bacteriophages was counted in various pH ranges (pH 2, 3, 4, 5, 6, 7, 8, 9, 10, and 11). Each solution was prepared according to pH using a sodium acetate buffer, a sodium phosphate buffer, and Tris-HCl, and then dispensed into 990 μL. The bacteriophage OPT-SAL01 culture solution was prepared to a concentration of 1.0×10⁸ PFU/mL, and 10 μL of the culture solution was added to each buffer adjusted to the appropriate pH, then left at room temperature for 2 hours. Afterward, each reaction solution was serially diluted, and the titer was measured using a soft agar overlay method. The results are shown in FIG. 4.

As shown in FIG. 4, it was confirmed that the bacteriophage OPT-SAL01 of the present disclosure was highly stable for 2 hours in the pH range of 4 to 11. As a result, it can be concluded that the bacteriophage OPT-SALO1 is a bacteriophage with excellent pH stability.

### EXAMPLE 6. INVESTIGATION OF THERMAL STABILITY OF BACTERIOPHAGE OPT-SAL01

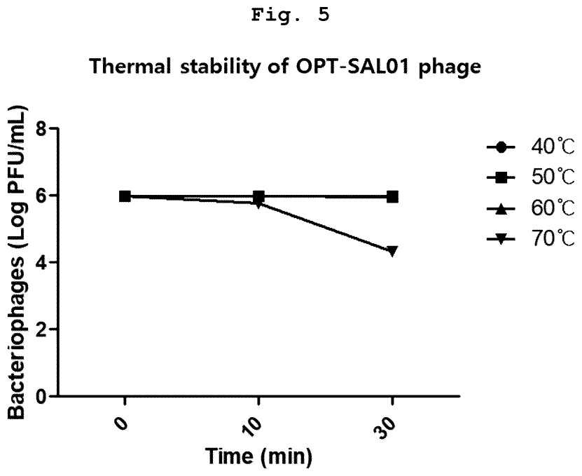

To confirm the thermal stability of the bacteriophage OPT-SAL01, the number of bacteriophages was measured after being left for a certain period of time at various temperatures (40, 50, and 60° C.). More specifically, 1 mL of the bacteriophage OPT-SAL01 culture solution (1.0×10⁶ PFU/mL) was dispensed into sterilized tubes and left at each temperature for 10 and 30 minutes. Afterward, each reaction solution was serially diluted, and the titer was measured using a soft agar overlay method, and the results are shown in FIG. 5.

As shown in FIG. 5, it was confirmed that the bacteriophage OPT-SAL01 of the present disclosure was stable in the range of 40, 50 to 60° C. for up to 30 minutes and stable at 70° C. up to 10 minutes. As a result, it can be concluded that the bacteriophage OPT-SAL01 is a bacteriophage with excellent heat resistance.

As described above, specific parts of the present disclosure have been described in detail, and it will be apparent to those skilled in the art that these specific techniques are merely preferred embodiments, and the scope of the present disclosure is not limited thereto. Therefore, the substantial scope of the present disclosure will be defined by the appended claims and their equivalents.

### [Accession Number]

### Depositary Authority Name: Korean Culture Center of Microorganisms

Accession number: KCCM13132P
Accession Date: 20220218

1. A bacteriophage OPT-SAL01 (Accession Number: KCCM13132P) with specific bactericidal ability against Salmonella enterica, consisting of a base sequence as set forth in SEQ ID NO: 1.

2. The bacteriophage OPT-SAL01 (Accession Number: KCCM13132P) of claim 1, wherein the Salmonella enterica is at least one selected from the group consisting of Salmonella albany, Salmonella agona, Salmonella falkensee, Salmonella hardar, Salmonella indiana, Salmonella infantis, Salmonella kedougou, Salmonella kentucky, Salmonella molade, Salmonella montevideo, Salmonella orion, Salmonella paratyphi A, Salmonella rissen, Salmonella stanley, Salmonella tennessee, and Salmonella virchow.

3. The bacteriophage OPT-SAL01 (Accession Number: KCCM13132P) of claim 1, wherein the bacteriophage OPT-SAL01 is stable at pH 4 to 11.

4-8. (canceled)

9. A method for preventing or treating infectious diseases caused by Salmonella enterica, comprising administering the bacteriophage OPT-SAL01 of claim 1 to a subject in need thereof.

10. (canceled)

11. The method for preventing or treating infectious diseases caused by Salmonella enterica of claim 9, wherein the Salmonella enterica is at least one selected from the group consisting of Salmonella albany, Salmonella agona, Salmonella falkensee, Salmonella hardar, Salmonella indiana, Salmonella infantis, Salmonella kedougou, Salmonella kentucky, Salmonella molade, Salmonella montevideo, Salmonella orion, Salmonella paratyphi A, Salmonella rissen, Salmonella stanley, Salmonella tennessee, and Salmonella virchow.

12. The method for preventing or treating infectious diseases caused by Salmonella enterica of claim 9, wherein the bacteriophage OPT-SAL01 is stable at pH 4 to 11.

13. A method for inhibiting Salmonella enterica, comprising contacting the bacteriophage OPT-SAL01 of claim 1 with a food, a feed or a container.

14. The method for inhibiting Salmonella enterica of claim 13, wherein the Salmonella enterica is at least one selected from the group consisting of Salmonella albany, Salmonella agona, Salmonella falkensee, Salmonella hardar, Salmonella indiana, Salmonella infantis, Salmonella kedougou, Salmonella kentucky, Salmonella molade, Salmonella montevideo, Salmonella orion, Salmonella paratyphi A, Salmonella rissen, Salmonella stanley, Salmonella tennessee, and Salmonella virchow.

15. The method for inhibiting Salmonella enterica of claim 13, wherein the bacteriophage OPT-SAL01 is stable at pH 4 to 11.


SEQUENCE LISTING (1 sequence)

SEQ ID NO: 1 - DNA, 86400 bp, unidentified
  misc_feature 1..86400: note = Salmonella enterica subsp. Albany bacteriophage OPT-SAL01
  source 1..86400: mol_type = other DNA
ggtacaccgatttctcagctactggcagtttagcaaactgctcccttaccttttagggttaatcttccattatttctgtaatgcgttcttcatctctggtgttgcaatagtgtcaattacaccagttttacaagcatcatcaaaccagtctggtaagatacctgctaagaacccattgatattcgttttaaggtactctgcaataagggcagcttcttcttcaatcatctgcacaacttcatctgcataagctgtaatcttcgagataccttcattaatcttactaacagcttggttagctaaatcagaaatagtatcaagacctttatcaatagcatcctgtaagtcacttaatacatcattaacgccatctagtgtgctgttaattgtatccatagctttttgaccatactctgtagcaacacccataataccactaaatggtgtacaaccaacttgctctcctgctgcacccataacattggagtagccttttgctacctgcatacgtgaggaaaactcatcaatagatttttgaccataatctgtcagtgtcttagtagttgatgtagtgcttgtcagactagttgtaaagctattaagcagaactgttgtaagtccagcagcaatgagcttatccttcattgtagggtctgttacagagctaatagagcttacaagtgatgtagaggcagctacggttccacctagaactgctgcaccagtaataaggggattagaaaaccctctaccggtttttaagagattaaaaatctccttaccttgttctgtcatctctttcattaagcacctttgaaattggtagtccagtgggatttgaaccctcttctcatgtttttcagacacgcgctttaaccatataagctacttggactataaattggggtgacctacgggatttgaacccgtatagaccatgttcacagcatgggtcattaccgtttatgataaggccacatttaaggactctcgtaagaatccttagaagtggcagcggcataaggatttgaaccttaataggacagcttcagagactgctgcattgccagttatgctataccgctaaatttggtactccatatcggattcgaaccgatacataacacagattttaagtctggcctctctgccaattggagtaatggagcattggcgggggatgttggaattgaaccaacttcttcgatttcaaagaccgaggttttaaccttgtaaactaatcccctttaaatctttactttcttagtggatgaataaagaatgccaacataactctttcccttgagaatgctctttcttctgggacaacacttttaagtttccatccaacataaattctccagtagaattgtttaccaaagactttaattgatgggataaaggcgaataatccccaagcattactgttccacattaggagataacctgtctgattatcttcagggtcagaacttacgttgatattacctttccacttagtaacatcttttacatctcttcctaacacatggtaagagaagttataagctttgtttctccagagccatccaactctctgcaaatagacacccagcttaccaatctttctaatcttagcccatcttttaatgtgaccttcatcaccatcaatagggttgtcatatgtctccatccatctgaatccaaaaggtagatgccctttcttctcactatagaatggaactacaaaaggtgctaagataactgctaaggttgctgccaatgggtctaacaaagctaggaaaatccatgaagcatacttcaaatatctcatctaaatagcctctttaaattggtgttccaagacggattcgaaccgtcactagtacaaggtttgagcttgcatcctctgccaattgggatactggaacgtggtactcactaaaggacttgaaccttttttcttcatcttgtaagggtgatgttttaccatataaactaagcgagtatgaaagaggtctgaagttgtgccgctaactcaactccgtggaatttgttacggtcttcagttgacaccagcggtctttcacttgacctctgaattggtgctgctcacaggactcgaacctgcatttccatccttacaagggatgtgtaatagccaattatacgaaagcagcattatttggagcatccagagggaatcgaaccctcaacctcagtttggaagactgtaattttcccgtttaaactatggatgcactaattggtggagaagcagggaattgaaccctgtacttaagtttgcaaaacttatgttttaaccatgtaaactacatccccaatgtctggtacaggtggagggaattgaacccatcgtcttactgattaagagtcagccgcataaccattttgctacacctgcattaatttggtacacctagagagattcgaactcacactgagcagattctaagtctgctgcctctaccagttgggctataggtgcattaaatctttttagagaacttctaagaacctaagcaacaaggttccacaacaagctgtaataaccttagaagccctctaaaaagacctaaataggtctttgcatagtcttgtttaatccggtgactagaacctttggagagcttgatattatctcttctccgtgacacctactaggtgctttacttgtgtacgacaatacacgagggataccaaagcaacatccgaatgaagcttggctttaaatcttattaggcggtaagtaaaggagtcgaaccctcaccgtatctctacagtggcaactgttttcaagacagtttggctaccattagccgctacctacccctaataagactttggcatgggacggaggagttgaaccactttgaaacggttttggaggccgttgctcatgccttagagtcttaacatcgtcccacactataattcgaagcttaccgtattttcgcaacactgtaaagccccttctcagattatttacatgttctggaaaacatgagatacagaccacctccttacacggagacccgataaggtcgtctaagtaatctgctaactaaattggtcgaggcggcaggactcgaacccgcatactccacttactcggttaacggctgtttagaagacagctgagatacgccccgtaattatctttaaagactctcttagaaagcccttaaagatgcccacattattaatcataccgtgggcgagtacgccaaattctttgatgagggattggaagacctcactggtgtttagcctatcaagctactgccagaaaaacattgtcgtttgcatttattttaaatttgcaaaatagacgctacgcaacgaaaactaaggttactataaagtggtcacatcccaatgtcaacaactttattgaaattggtactgtgagaaggattcgaaccttctgcctttcggttatcagccgaacgcacatccatatgtgctttcacagcattgttaccccttcgtgtacttctctgtggcaatttgcacaaagcaagacacacttatcaacttcagactgcattgtagccagtgagtagtgttttttacttactgtgaagtcttttgtcttagggtctaagtggtgaaactctagagcctttatgcacctattgtaaccacaaattgaacaacaaccacccttgtaatcaactaaagccttcttcagtctgtcacgagactttttaacgtgcagtgatttggctttgctttttcatgatgggtcttatgttttttcatgactccacctgcaatacattcgtcacagtattttctcgcttttggcgttacctttccgcatgaatggcaagagccttcctgacttctctttgggtgtttcttgttgttcacttttgcagaacaagagcgtgagcaaaattttccacctttaacggcttcatttccgcaatctttgttagcacaaatcataagtatcacctataaattggaggttcagatgggaatcgaacccacattcatagggcttatgagacccctgcattaccttatatgcgactgaaccatattggtagaagtggagggattcgaacccatcgcctgtcagattaaaagtctgccgcatcaccattctgctacacttccattaaattacctttgcagtaacctacgcagaccttgctaaagggtctgtagagtcatttcctattaagctaatctagaatcatgctaaggaaattcatcggcactcatgggacgacataattagccacataagctactgcaaagataattaccagaccgttgtttatcatcttaagtgctaccattacaccaactcgacatctgccgagtgaaggaatcgaaccttcgccttttctttaccgagaaatagattgtttaagttttgctgtaaacggtcttctgtaaatttgttgagccagaccaagttttaaatttttcaaaattgaattggcttgaaaatatcatacgtacttgctgcaattggtcttctgctcctttaaagaatactcgtaagaactctttaagggaggggctaaatagccctctcaaaattggtgtgccgtgtaggagtcgaacctaccgagtctcaatgacaagggatttacagtccccaccgctaccatctacgggataacgacacatctaatttctattcaggctaaggacttgcaccttacaaatcggtgagcaatcgcttctctcctaaattttgcggttagtcagggattcgaaccctgtgccattcgcttaacaggcgaccgcacataccttatgtgcttcctaaccatgtttagtattaccagaccgtatctttcaatttttccgctaaaaatttaataaatattgctgtaaacggccttctgtaaatttggaggcgggtgcaggagtcgaacctgccgataccatgctaatgagacatgtgagacgccctttctctatacccgcaattcttaatgaccagaccaaatctcttctttgtccgatttaccttcagatgaataagattaagtttgctgcaattggtcttctgtcaaaactggcgttccagaagggatttgaaccctcaaatatccactttgaaagagtggtgactataccattttgtctactggaacattaatttggtctctgttggaggacttgaacctccggccttaccgccccaaacggaacgctctaccaagctgagctaaacagagataaacttttcaaactctatgtaaccactttaacatcattttttagtggttgtcaagagcttttttaaaatatttttcagtatcttagaaaagctctcatttcgtttctatgtagaacatattaaagggtatcaaacacattgtcaataccctttttaaaactttttaccagatataacggtcaatcattactgctttgagcattacgctgattgggtcaaacttctcaccaccaagcagagcttttaaagtggctggagaaaaccctgataccattgctacaccattatccttaacagatacttcgcaagtaccattacggtttgctaagtaccagaataccagttgtggcatttcgtatccagcttttttgtacttactctgaattgcttcaaagtttgtacgaccgttagctccatcaacctgattaaactccatatcagagaagataataagcttacttggcatatctttctgagtcaagttgtttcgcttacctacctcaagaatgcggtcaaaagctgcttgtaagttagttgagccatattcaacatgacgcatcacctgacgatgacggtttcgtaaatcaccactcagttcaatgaaatgagggcttgttgaatagaccattaactcatctttaaagcaaccagtattacgttctgctacatacaaggcaagtgatacaccaatatcaagggcagtgattgaaccaagattcacccaagacattgaacttgaaacatcagtcatacacaagatgttttcaccttctgccatccagtttggtagtgctttccactgctcattagcaacgtctgcattaccatacttgatagatttaatcacatcatatgggtaaacagcaccagcgttaatcttagcctcaccttttgagagtgactcgatgtaagctttgtaacgttccccatctttacggttaaagagtttttggtaacgtgcagcagcgagtgaaggaatcttgctgtagtcaatcttaccaaactcattagcagagattttttgttcaaccgtatcagacagtgcagataacagcgtgcgatactccttctcgcttaagtttgcaaacttacagaaatgttttacaaactgtttgtggcgtggttttactcgtggtaaccacttagccgctaaacctgctgttgcagggtctagtaatgctgcttctaaatgtttgaaggcatctgtctcaaaacgagtacctacgaagattttgaagtcatcaaaacgaccgagttctgcaatcttatccataatgcgaagaacctgtgtaggttctaaaactttatcttcaatcgcttgaagtaaaacagttcggaaagctttacgctcacccataccttctcgtacatctcgcatatgcagtaaaatacgaactgcaacatcaacatcctcacgcaaagctttgtagaacaggtctggtaagatttctacattgctacggcttgagccagcggctttgtagaagtctaccagagcagacattgatgaagtatggtttacagcaccattttcagttcgacctgtatgaaggtgcgcatgtttaaataattcgctcatattttactctcttctctcatttgttgtttgatgtgacagactttagagcaactttgtagagtctgtcaacaccttttaaaattattttttgagagtcgctacagcagagaacgttggtttgttatctgtctgtgactgaccaccattatcaggagttttcttagtgtctgctgcaagtgctttcatctcacctgctgagtgagtaaagatagttttaccgacctcaatcatgctcttgatggttacgtcgtctgtgtttaaaccgaactctgcaagcttagcagcatcccttgtaacaatcgcctcaaacaactttgcagcaaactcagcagaattatcaatggtcagttgagcttttacaagtgagcttttgttacgagagccttttggtcttccagatgggtttccagactggccttttttaaactggcctttgtttgttctgtttttcattggtatgcctcttataagacctcttaaaaggcttttaagataaaagatagatatccagaaagatatctgtttaaatagcctttttagaggaaccttttaagttatcttctaagtatttataagcctacaccttgtcaagtcctttgtcaacaacttttttaacttgcaatagttcttgacttgttgtatggattactgtaccatcttacttaaagctgtaggtctgccttgttctacaaaggagacttaatgagaaaatcaaacaacccgaagaaaggtaaaaataccaatcactgtaaggaatcaaaaagggtagagttactctactattcatcctctgaaattggtctgtacctgttctttcaaaattacagaagacaagaggattatctatgtgtagttcccaattagaaatcgcagatattatagatttatatcaaactgcaaagagtcatggctatataacctcaattggaaagaatagtcactacgatgctttgactggaatgtatttcagggcaatggctcaatctagtgaacaacacttgatggtttcttccagtgagtttacttcgtttctctattgcagcaaaatcataaatcgcaggagaactgaaaatgttaacagtaagttttaattacaatagtgatggctctgtatcaattaattcaccatatgcaaatgacctattgaaagagctagttaatcagtgtgatagaggtcttcactatgtcccaaattctttcaagcagaagactattgctaataacttgatgcgtgtgacggttacaacatcaaatccaaactacgacattgatagtgaaagcccttattctttagtggctcttggagagtgcagtcaatttaaacttgtatgccacgactcagaaacatttcttaaggtgttttccaaccttattcacaataataagtacgggtatgttgacggtagtgcaaacttctatccagcaaactatacatgcctattgattgataatatgagaagtagcaagcaagagcctacagaaatcccatttgatgtgaactctagtccagacgcagaaacaagtaataactttgatatgagttacgcgctatcacttagtaagaaatccgagttcattgattatgtcaatggatttggttttaagtttgacgagaacatgaatctcaaaaaacttaagaacctacttaagaccaaagcttaagtataaacaggggctaatgcccctttatctatttataaggatatttaatgaaggctaagagtgcaaaagacttttattgcttcctacaatcctatatcaattccgtagagaaaggtgatagatataatcttaaagatgttattgcatcacctttaacatggagacttagcaaatggcctgaagaagatatcacacccactagtaaacaacctacctacatcccagatattaatctcccagattcagagaagttattgtacccaatgttccacattgttgggcttggtacatttcttatggatatccagtatgtaattggtaagggttataaggttgaaggtattgttgttggtgatgtatctccacaacataaaggctattttagattaaacgcacgtttagaggctaaaaagaaatgattaaagcaaagacttacccagacttcaaagaatttgtaaaaggtttcgtcgcaaatgtaaaggctggtaagagatatgattttagaacatatcaagaagctattttaccacttacctatagctcatattggcctgaagctgatattgcagaggttaagaagtttgactacaaaccagactacaaagttcctttcagtgatgaacttctttacagtgttggtgcacagatgagaactgctgatttcttcatggatttacagtatgcaatcatcaatggtaaagatgttgacacagtgtattgtgaatggttggcaagagttaagccgttctcaatgttgaatgctaagttgaaggatgccattaagccaccagtaattactcagcagccaacaaaccaaacagtcaatgaaggtggaacgcttactctaagtgttgttgcatctaatgccactggttatcagtggaagaaagatggtgagaacatctccagtgccacttctgcaacttacacaaagcaatccgtagcgcctgctgatgctggttcatatacttgtgttgtatctggagagggtggaacaagtgtcacctcagacgcagcaacagtgactgtgaatgccttaccagttatcactcaacaaccatccaatcaagaaatcactgaaggtgagaccttaacactaagtgttgtggcaacaggtgcaacaggttaccagtggaagaaaggtgaggaagtcatcccagacgcaactgctgcaacttacaccaaagaaggtgcaaccgctgctgatgcaggaagctacacctgtgtagtaactggcgcaggtggctctgtaacatctaatgcagcaacagttacagttaacccagcaggggaggcataatgcaactctcaagaaaaggtttagaagctattaagttctttgaaggtctgaagttagaggcttacgaagactctgccggaatcccaacaattgggtatggtacaatccgtattgacggaaaacctgttaagatgggtatgaaaatcactgatgaacaagctgaacagtatcttctcgcggatgttgaaaagtttgttgcagcagtgaacaaagctatcaaggttccaacttctcagaatgagttcgatgcacttgtaagtgaaacatacaacatcggtatcacagctatgcaggattctacatttatcaagcgccacaatgctggtaacaaggtaggttgtgcagaagctatgcagtggtggaacaaggttacagtcaaaggtcagaaggtcacttcaaacggtctgaaaaacagacgtagaatggaagctgacatttaccttgacagtgtatatccaaagtaatatcttcataggctccttcgggagccttttttattttctaaggagaaaattatgaagctttgggctagtgactttggaacttttaagtacacacgtaatggttcgcttgtacgcattgtcggaaacaatgtggtttcaagaggcgacaaggtgtatacacggtttactgtagagcttgttgaactgtcacctattgagtctgtgaacaatggcctgttcaagtttgagacttacaatgtcaatgaacatggacaattcaatcctcttggtgaaagtggacttgatattatttcagagcacccattgacaaaagagcaacttgcaggttactataaaactgttctggaaaggcagttagcaacacatgaacaagaagctaactaccatttgcagcattgcgaaactttaagagcaaaaatcgaacaagcagagagaggtttttatgaataaagatgttagaagtattcaagtaaaggtggacacgtttggccatgatggtcgagaggacattggttttgctgatgcagtaatattcaagtgcaacggagatttgttcctttgttttacagggtctggaagcacccttcatcgagtgcgagatgatgaagatggctgccctgtatttggttggtttgacagggagttccctctttacgcaatccactacccagatggtagtgaagactggacgacccaatcaatccttgatgagcttaacggtgttccagttgagcaagaagaagaagaagaagagcaagaacctattgagctaaccttcaccttcatcaaagaagagactgttggggacttatcagtaactgaagcaattcaagtcacacaggttattcgttaatgagtaaagtacagattattttccctatttgtgacttctcactagagcgtgaacttgacctgtacgaagaaattactcacgaaatcatctggtctgttgtagaagaggctatcaagaaactgtatggtggcctcttaaatccatcaagtaaaaagctcaacactaaacaagtagctgatacgtacacttcatacgatgcctacaacaaaccttttgagaatacctgctttgaccttatggtaggtaacaataaagtcaactatttcttcgtcagagagtttaacgatgagtaagctccatgtaacggtctataaaaacttctcagatatcaaagagtctttaaccaataagcttgacatgcaacggaaaagactctttttaatgtatgacattgacaactataaccatcctaaagagttcaactacaaagatggtacaaaggttgttgaatttgaagattctgtaacggtgtatgtcaaacatgatttaccagcaaagtacatagggatgttagagtactatatattcaaacatacaggtatgcgtggtgaatctgtcaagatatcttctatagaagtttttgagaaacctaacacacaacttaaaaaatatttaatgaggaaactgtaatgtccgaagagcaacaggatatcatcccacaagtaacattagtccaacattttggaaacattgaagggtgtgttgcactttttcaaccaagtatcaattcccctgcaaaagtttgcaagttgactatgaatgttaataacattagtgtctgccttgttgatgaagtccagtacttcaagtttaatgacagagaagttgatgctgcacttttgaagtatcgagctaatctcgaaaaagacattgaccataaagaacttgtaacactgtttggtgaccttcacaagcttcttgaaaaggttatgaagcgtacatactacatgaataatggctcaattatcaccaccctaatttcaccgtgtatctcagagccaattctgaccgatgatggtgggtactatgttgtagcatcagcagattctgactggtggatgaaaaatacagcactgaagacagtgattgaagccattcgtgagcacatcccttcattcagtccgtggaaagggaaaggtgatgaatttatcgcactattgagtgaagagagtaacaagcgcagcgcattactgcctaaaaaatactcttgaccaagtatacaatattaactatgatgggagctatcgagaggtagttccctttttagtttctggagaatagatatgcctaaagtgaaagaacacgataaaattattttgtatgttaaacagaacccttcaaagagtgtggaggctgttgtaacacatatctcgcatactggcacagtttattttcgaccactgaagaacttagactttgacataacaccaaaccacacatttagtacctccgcaagactgtgtgcaggaactgtatactcaactgctgagattgagtctttagctttcacaggtgcttacaagaactttatagggattgctttaaaggactctgaggaatcttttaaatttttcatggatgctgccaatggtggtcgtagatataccatgccagtctttaagaaagttgatacagagattgtcaacaagtgcgaagaaattagcagtgacctgaaggaacttaaggcatctgtgcagaatgcaatttacattattgaaaagaatcacttagaagttcttttcagagatatgctgaaagcagggtttactccagacgaaattacaagagaaactcaacgtcagttcagaaatgctatggtgaaagaatgaaaaagctaactacagttgaagattactacagactgtctctattagaacaataccgtagaagtcagaatatcagaaaatgctatggtaaacacgctgaaggtgacttttaccgttgctacgatgcagatttaaaaggtgtaacacctagagggaaagtcctgcaaaggcttgttgaccttgagtgggacaaacgattgagaggggttgggaaatgatttatgaagaaagatataagatagattatcaggatactcgtcatcatacttctttgagagtaaccaaaccaaatggagatactggtattatagcacattttggtggtgattattggtatggtacaggttgctttgaaggctacaataaagaatacttgaaagcattctacagagactttacaaacgactacaacagggttgttgacgaaaagaataagtgcattaagcatgaacaccatgccagaggttgtttaagtacggtaatggtactggcatttttcctagcaacattactggcagtgttagcaatcagttccatagctcaagacttaaccattgcacagattactgcaaaggtatatgatgtttggtacttgtatgctgtccctttagttggtatcatcatcgcactaatgagattcagagttcataagaaacgtcttaaggattctgaggttaaacttgaagaggtaagtaaagaatgcaacctacaattatagctgtatgtgttcgttttgcaatcgctgaaatgattaacaaggcaatcttaaaagatgcctatggagaaactaagtaatgattaagacacctgtaccaatttttggatttccttctattgaagagttcaaagtttatcttgacaaaaacttctacaatgagcagcctgttactctactgaagagtgacttatcagagcttcttgatatggttattaaggcaacttctgagaaggagcctgagcagaaagctgagaagaagactagcaagaaatctgaaaagaaaactgaaaagtctgagtaataacttgaggggttacttgatagcccctttgtagaaacttagaaggtagcgaaatgaagattaaagaagttgttcaaaaagcaatgcttgacaactcaactaaagatgaaatgtacaaagagatttgtgataagttgaattgttcaagacatgctgctaaggctcttgtacattgctttatctgggaatgctcagaagcttatatgcaatatgtagcttttggaagttctcatttactaggtgatgtagaagttggtgagaaactaaaagaacctgagatgaaaacagtccctaaagttggtaacgtataccctcttaaagacttcaagactggaaagattgttgcaaaaggtgtagtagagcatgtttatgatgatggcgaatacttacttaaagtatttgagtatggtagccactatacacacttatgtgggcttacattctttgtaacaaaagaagacctcattaaaaacaaagacaacaagtttgctgttccagattaccaagttttacgataatggtgtgatagatatggaaaaagacaacttaaaagatgttgaaggcaactatctagtccttgactggaatgatattcgggaagcactctccgaagaaaattttgattggctccaacaaattatctttgcagtacgtcataccagaggtattgtaaacggtaaagagccattagaaggaatctttgtagagaagtcttatccgttctatgaagacactttgcagaaagttaaaatgtactttagacagaagaaccgtaaagtggtcactatggtgtctcttggtggtcagagtatgtctgtgatggaagatattaaccctaagagaccgagtaaaggtatttgtataaaaatcacaggtcatgaagagtatatctcagtggatgactttagtgcattctgtaatggttctgctgtctttaatgggttccactatgatatccgagtgtacccaatcaagggagatgatatcatgatgcagatatttgataacaacaatggatttactcatttttatcagacaaccaagtcatcactgaagacagttctggaaacactgatttaaaatatcttaagagcctccttaattgggggctttttaatttttataaaattttatcggttgtcccaacctctgggtcaccatcatatgtgaataaaatatctacccttcttaacaaccttcttaagcttactctctatatatagttatatagtactatatagtatcttagtaactatctagtggtcttaatagcccctctgtataagccttctaaacactcttctaacaagccttcttaacaacctatacaatcaccttaaccatctctctacaccatcttagaagcccctatacacctcttaatctctaggttacctgtgttacttaacagtaacccattaggttagctatgttaccctctataggtgttcactaggtattactaagcctctatatagactgttttaacaaacttaacaggaatatctttacagaccttatctcacaaggtctaacacggtactaacaaggtatcttataagcctgtttatgcctgtatttttaaaagactgtctaggtaattattcagaattttaaaatttttataaaataggactatcaagtcattttttagggtctttgaaggaactgaaaaatatctgtagagttgattcggtagaatctacctcatgatgtatatctgcttggtagcccttaacatccctgtaacattccctttgcagaattttaaaatatcattaacaatctcttaacagttgctacatcttcttaacatctcttcagaggtcttaacagattcttaacagttttaacaagagtttaacagttttctaacaagtgctgaatagtctttacagatgcttaacagatgtctaacaagagctaacaagttaatttttaagagttattttgttaagataattaacaatgttctaacaagtgtttaatctttttaacaaactattaacatggtcactatctcttttcttatcgttccctttccctcttagtgattctttaaacatacctcttaaaagctctctatagctctttgtaagctcttttaagtctcttcctagtaattacaccccaactatacagatagctatttatagggctttacaggggctttaaaggctatgccaaagactcttacaaggcttttacatggtcttaacagagatttaacaaggggctaacaagttgctaacaagtgtcttgaaagcataactatgtaggatttgttaagaaattgttaagaaaatgttaagggggctagcatttacgtaacttttaagctacttcaaagtaactttatagccccatataaaggggctgtaaaggtatctggggctatttagccccgctgcgtcgattagttataatcttgtttgttgtagtacagggagacgattttttctccccaatagtgaaaagtgtaaccatctttaagaacttcctgttttgtttctttccagaactcatcagaaggattatagtctttgatggtctgactgataagacctttcaccatttcaaaggcgttgtcagtgcctacaaggtcaaatttaacatccagagagtagaagccaaattcgttaacagttgcgcggatagttgccatttttgaatctcctagtttaaggactttgtttagtagggaggtcatccctcccgttgattagtaatttacttgttttgatagctgattgcaagcaattttttaagtttatctataaagaatcttttaagttaatcccttttagataaaccccctcgaacaaagttaccaatgtcaagagggattgtcaacacttttttaaaagttttttgtaagttcttgattaactgatgaatattttttcaggttttacatagctgatacgctgactttttaggaggtcatcccagattagcgcggcaatatgagagtaaccggattcacgtaagagcctcattgctttacttctcgtttcgaatctggttgcatagtactcactattcaaaacagttttattcccttcgttgtctgtatgaataacctgcattacttgaaaggggcttgtttcctcacctttgttccattgctcaaagtattttaggcactgcataggtgacatcttgtcacttacttgagttgaccaagatgaatcttctttattggtgattacggttacataagccatttttgaatctcctagtttaaggactttgtttagttgggaggtcatccctcccgttgattgacaatttacagacttttatcaggcttgtctagtctttttcaatgataatttcatattgaccatcaacgctgtcaaaataaacgccgtcgctgtcttctgtcttaaaataacactgaatgtcatcactggcggggtagtcttgtcttgctatctgacagacctgaacggcattttctttacttcctgtaaactcctcaagccctttaaaatcacatgaatgaccgttacaaatagaaaggatgagataaacagttgtaagcatgatttaagcctcttaaaagccccttacaggggctgaatagtgtttataaggtgattgtataggttttcagaaaaagctctttaaatctctttctatgatgtttcccgtcagtgtgtccatgatgtcacaaaaaacttgaatactgtcggactgttgaacgcttttagcgagaagttccagagactcgtcaatatagcttaaattcgggttatccagtgaaggcagacaaaatggtgaaaacatagcattaaattcagtggtgtaaacctttttaatccagtttaaatcgaaatattctgtaaaacttgccacgccagacatattatcacaagaataatcatcgtttaactcatcttctggaatatcaagttctaagataacaatattttgggttttcatctggaaagctgcggtgatagcggcattgcccagagcttgctggataccttcagagattatctcttcctcatcgtcgccatagattttattttctggataaacgtaaaaattcccatccatatccgaacaattccatgcacctgatggcttatcaccgttattaatcaggttgataaaattttcttgagtagtaccgtgaaagcatttcatttttgaatctcctagtttaaggatttagcggtaaagctctttatctggggcttactttatcgaactataagccccttgtaaagtactttatccgttattttttacaacttttaaaactgcacgatagcccaagcaataagaatcaccgaagagagaccgacccatgaaagaatcctctttgatatctttcgccgtgacggttttttctacgccgtgaaaaaggactatatcgccaactttaatgtctttgatgtgagtagtttcaatgtttaagccgttaataatagccattttttgaatctcctagtttaaggactttatttagtggggaaggtcattcccgccccgttgatgtgtaatttaatctatctgaaaaccagtgtcaacaaaaatttttatagttctgcataaattgcatcacagatattacgaaaatcgtgatttgttaagttacgttgcaaccatgttgcgaaaaagtcagcgattgcaaaattagcgtcatactcgacagatagccacactttaacagcaaatcgaaacattttcaaatcaagctctttattcattaccttagatttgcctaacatgtcggtcatttcatattgtgcaccgtctagcgtgatacctgctgcaacgtcagccaatgagttttttacgttttcaacgtctaagataacgctttgaatcttcccaataatgttgtgcaatgtttcgtccggtgctgatgacgtaataacattaatccggttaacggtcaaacgtgcttttaagattgataactcgcgattattaatagccatttttaagttctccaatttaggatttagtggtaaagctctttatctggggcttactttatcgaactataagccccttgtaaagtactttatactaaatgcagctttttgccttttaccatgatgtaacttgttgcgctattgctatgcacatcttgataaattctgcgcaaacggccttcgtagtatactttccaactggttggaatctttttcccgtatcctgtagcagttttttggagtcctgcaagttgccaatctaagatatgttgttcggcttgtaaatcctcgaagtgcattacaccgttcacatagtggcgcaaagttacatttagagtacgaccgttgttagtagtatacattttctaatctcctagtttaaggactttacttagtggggaggtcatccctcccgttgattagtaatttacttgttttaaaaaccaaaatcaagagaaattttttcgtttgctcaattcttgaacaattacagcgtagtagtcttttaagtcactgatatttaaagcatttatcagtgaattataatcatcgactaactgaaaatcatctttgaagatatcacttatctgataggttttcgctttgaatctgaagctcatttctagccgctttatttgctcatcacttaaacgctccagaaagctcattacaaggctattattgacctgttgcgtgattgccttacttgccagattgtaggcgctgtaagccttataaagtgctataatggcgatgattgcagtgatgcccatcattagcatggtgttacctctctgcgtgatactttaccatttacagttatctcataagcttctaataaatcactttcagatttaacaggacttttaaaagtatcatcataaacattaatttgcaatacaggctttttatactctctgcaactattccataagcaaattactacgcgacggccttttattttgccgtgtaaccattccataacatcatgactatttcttgatgtctcgataaaagcaattttattgagtaatttgcttagattttcacgcacttgatttgataatttcatttttgaatctcctagtttaagggctttatttgataaatctctttatctggggctatcctaccagcttgttagccccgtgtaaagaaatctatttagttcttaaagatattcatcactgaatctttcaacgattccaaacgttctaatacttcagattctgcgtctgcgttgttcacctcttcttttagtctatcctctaaatcatcgccgtcacgatagcaccaatcgaaagcgtaaccaagataaaaactttctaaaaccgtgctaccgtttgcatccacaactttcgcggcaaggctatactctgaagcctctaaatctctttctagttctttctgtaagcttgtgtaagcctctttcgatggattctcacggccttgttttgcatagtcacttgaaagctgcttaaggctgtaatgcaaagggattgcatatttaaactcattacgctgacagttggtgtttttaatagttacaccgccttgatggtcatctgtaaactcccaattatccatattatggcttgcttcgtagcaattaacagcctcataaatgatggaaaatttttcttttacaacttcaaaggtagcgtctacagttttcatttttgaatctcctagtttaaggacttgatttagtggtaaagctctttatctggggcttaagatatcaacttttaagccccttgtaaagtactttatcagattaaattgccagttaatgataagtgcattttatagccttttttggtaaaaacgttcactttaccgagtgattttacacctttaaaaaggttgtttgaactgcgttttaaggttagttgcttatcagctttcacgccccaccttacagcagtttgttttccgttgtcaaccacttttccgcataactttttagccataacgcgagcgtcatcacgagtctttgcagtaattacaacgtttttgataatagccatttttaagttctccaatttaggatttagtggtaaagctctttatctggggcttactttatcgaactataagccctttgtaaagtactttattagttaattttgaaaattgtcacataaccatcgctcccgatgatttgtgataccccagctttttctacttgatagcccatattttcaagatggtaaacagcatcatcgtaaccatatgcgccaccctgataacgtcgcttatcacattttatcaggtttttaccgcgtccagcaagtgcattcagtacggctttttcacttgatagtgtaccatctatcatggttgccgtgtaatggttgattttattcccattaatatcatacttaaaattgaatgcgtgaacaataatcaatttaccgttaaatttttcatcaagtgacttctccagtgcgttgcggaagttgtctttattgatgaatttcggtgcacgataagccattttagaatctcctagtttaaggtaagttagtaaagctctttatctggggcttaagatatcaacttttaagccccttgtaaagtactttttatatatcgtaatagtaaaccgctgtctgttttatatgtcgctttgtattctttgttaggtagcaattagagttaattgccgtttctacatagttgcaataatcagcttctgatattctacgccatgcgccgcacacttgcatataaaaacgttttgcctttgtgttaacgttggttgtaaccatcaattcgcgactgtacatcattttggattctccagtttaggaagtgggcttgcttgcccgtctgatgcaaatattagataaccatactcaaagagtcaataggaattttaaaaatatttttgcaagaattgtaatgaaacgggtgcgcgcgactaccacaaaacagaactacaatcaagaattatttttcaatccgctacaaatttttctgttgacttttatttccagatgtggtagccttgtgagaaatggcgacattcccacctgcacccacctacctgcacaattctaaaatgatttttcttgcccgacctgagtgggttctgcacgttttgaaaatgaaaaagctctttggagattacctgcacagtttgaaaatgaattttctctttggagttcaaaattctctctacagatttctctacagaactctacctgcacaattctaaaatgaaaaagccctccttacctgaaaatttaccagataagaaaggcttagtctattgtgaatattctacagagtttcttagttctttgcgtctaagaaggtaacttccggtgaagagaaaatttcctcaacatcctcatcatttcttgcatagatatatgtatctgcatcactgccaaatgttactgcaattctgtgattgctcttcgacaggtgggtaactagagcgttacttagagacatcttttcaaccggagggttactttgatgtaccttaccttttcttcgtctgaagagggtaactgtgttatctgtgaacactactacacctgcaaagtattctggttcacagtagttcttccaacctgagcgtaacttgtggtacatctctctttcgtagacatatagatagacatctacactcttcatcacctgattaatgataacggtgacggcttgagggtcaagtccaccgatacctgcaccaatcataggtaaaccaactttcttcaactgattgacttcacagtatctgttaagttgtttcaaagatgactctaaagcactgtatcgggcatctttaccagtcttaagctgagtgtaaaggttagctatacggccttgttctaaacgtgctacagagaaattacctagtaaactttcacagggtttgtggcccataccgcctgcatacagataaacttctgtatcagtctcatatgcttttgggtaaagtttagaaatcttatcagcgatacctgcacccattaagttcatacagttgcaaccgtgaccaatgatatcaaacttacctttatcaagtgcggagaagatatccccattgattatttttacaatacccatttaacatctctcccaagattctttatcttctgaaggttttacacgttgtccttcgtggtctacccagatacatccacaaccttcacagattactggcattgcatagccagctttaaagtcatcttcagtaataagaccttttaagtctcctgtatcacgtccgaacatttcgatagcacaatctttgcagaagtcagccataaatatacgccttatagttcaatttctcgtaactctttaccttcatcatagaaacatttatagaatgtatctatagagattctggcaaaggtgaggtcttcaaagtattcttctggagttatccctgtaaaactaaaaccatcaaggttgtaaaacggtatcactgtgtcatcgcttatgaaaacacatgcagatatgacttctccagtgtctttgtcaactacctctagagctttcatagtttaccttctcagttagctcatacaaatcaacatgctcaatactatcaatgttgataccaatctttttgaagcgttctaccacttcgttgtcagtcatccatatcagacaccaatgattgtggatggattcttcaagaaggtcaacacagtctttaaaagtttttgtatcacttcttgttccaactactgcgattgtcccatacaaccacttaccattgccataagatacttgtgctacgccaacttctttatctttctcgtaataaacggctacaaaatctcttccgttaatctgcatttgtaagttcctctgttggtaattcaccattttcagcaaagtactgaaggtatggcaataactctttcacctgttcttggttgagtttcattctttggttacctacaaaatctttgcctactaatgctgggatatcaagtgttttccagccaccatctaggtactgaacagagacctctttagcacctaaccagatacatggttcatatgatgaactttcttgtaaagtacaaatctctcctttagcatctttaaattctgcataagaaaatccacgatttgttggctggaattgcataatcatttcctcgtttttagaactgctaagatttctttagagtctggtaagaaccatgtgtagtgattctctttgatttctggatgagtgtcaacctttattttgcaatgtttaccaacatgcttaagttgtgctgtgacaccttgcataaagatatgacacattacattatccataatggttggtgaaaaatcaacccgcatataagtttcttttggtcgtggtagcagtaagtacccttctacatagaaagtgtcttcgttctctgtcattgctgtttgttccctaataaaatcatgtcaataaggttattagctttgattgtgcactggtaaaaaccatcacgataaaactcaagggctgctgccacatcgctgccagtattacctttgacattgaatcttgattcttcttgagtgtacttgtcactctcacttacttggaagtttttgaatgtactgttcactttgttcgttgaacaccctgacataattatcagtaacacaaacattataaaactctggcttagtcttttcatatgttagtacctcttttgtatgcttattcttagcaagcacatctttcaaatcatttttgtaattggtgctcagtgtgactaaaccttcctgataagcatctttagcaacactgattagtttctcattgtttagctcagcttcagcaacttggtagtctctgtaagagtatcctccccagacacctgccccaactaagaaaacaattatgaaagtagcttgacagaactctttaaatgtcatttaacaacctcctgaaatgaaaaaggctcccgaaggagcctgtatcttaaccttaactgaggactttagcaaggacatttgcagccatcgaagaagcttttgcagcagcagctaccccagctttgacagtgctatccttaatagcagataccgttgcagcatcagtaaagagataagtagttgttttagaaccacttgtaaaggaaagcataagagcagtttccagattgaaagagcgatacggagcaccttctttaacgataccttcagcacctaactctgaagccatacccatatcatagatagtcatcaggttttctttgtgtgcagtcgtcgaaccagcacctttgacatgttccttaacattcagaagagcacgatactcccggatgttcccgtctgcttttacattgacagcacggaagattttaccctcaaagttgcttttgataatgttacgaactacttcagatttgttttccgtaacgtccagtgctaccgtaatgatattgttcataagttttctctcaatgtttaactaacatggttttaatgtgtggaaggattctgtttaccttctgaacattcttctctgtatcttctataaaacatactatattaaagtaagggagaatgcaagactgaatcatcctcacttttaaactttctgcacttagagagttagttccaaaaccacgcataaaaagcatgtaatcatgctctatattgtgcctcaagaacatctcagtaggtattctctgagtttcacctcttgctgttaagaggccaatggcagcatgattagcaattgcatcaatgatattgaaaatatatgttggttttgctcttacagagtcaagaagattagtgtactgtgaaaatgacccgtcagtcaactctgtggagcaatcttcgtgattaaaatttgtaagaactccatcaatatctgctaagatgaggttgcctttttctacaaggtctttattgactatgactagactgtcacgtcttacacctatagattgtccagtctctttgtccacaacgttcacgattggttctgatgtgtggaactcttcaagtgaacatacaacttctcttgcagtatcttcaaaatccctgcactgttctggagtccatacacaggaatcaagacctaactcatgaggatgcaagtcgtaagcatggcacagtacttcatcaaactttcttaattttttcatgatttaccttagaaatgtttgttgcaaattcacattgagcaatgactttcatattgttatagacatcctgagaatatttcttagctttggggattttgtaagaatacccagcattatatgacgctaaaactttttgtaaagtcttctttgactttggttgaccatgtaccttcgtccaaaactcaagctctttgtgtgtttcctttgcagcatagtcgaagtcttttaagagtttctttttagccacattaggactgattttgttacgctttacaacagtcttcaagtgattctggaagataccatagtcatgggtctttttattctccatattcagacctaactctgactcttgtaaggctatagcagctagagtgatgccccatcctttacccatattattctcaccatactggtaagcttttaacatgtttactttttgactaattgatagctctgggcagtcaactgcataagataagtgtgctgtaaacattaagcacaaacctagaatcaacttcttcattggttctcctgtttagtttatcgtgacctcaattatacagcactctgcacaaaatacaaataaaaaaggccgccgaagcagccttttaagaaattacttattagatttcacgtttacgttgggagatgagttcaccagcagtgccaacgatgacatgcaaagcctcagtcttaccatcccacgctttcagtactttacgctgattatcaacgattgctgcaacttcataacgagaggaacgcatcttcatatcgttgtaatcagttggaacagatacaacatctcgtggatggacacgaaccttcaggatggtatcacctgagaagtaacgaacataatcccaagcaccgacatgaagaccttgagagcaagttacgttacggttgttatcaaccatccaacgtggcatctctacaatgttacccaaatcattaggtactttgtaggtgtgagagtcaaccagcttgccttcacgagtagagactttcttccaaccaatgatgtaaccttcttcatcaatttcaacatcaaggtgagatacaaatccccaaagttgttctacagagtctttagatgggttttccatcagtttttcgaagaacattacaagtcgttcaaaacctttatcaccagtcttcatcatgtgaaggatacggtcaaccagagtagaacgcatcccaacagcaccgtagaatagcttatcacctttgattgtgatagctccctgagtgaagttttcgatagacttacgaatgttcatcagttcaaaggctttcttgaactcacctttcacaacatgaactacaatctcttgatagttcggatgagtagactcaacaatttcagattctgaaccgtaagtcataattacagagtcgccagtaatcatgtactcaatcttgtcatctttctgcatagcttcatgcagtttatttactggaactttctgtacagcgtctttaacggccttttcaactggttttccagctttctttttaactgacttagcaacttgcttcaaagtcttcttagctggcttagaagttgcctttttcttaccttttaaggttgcttcatgacgctctacagcacgaccaactgaacgggttgaagtattgaacttctgtgcaatagctgttttagtcagcttaccttctttaaccagtttgtaaatttctgcgtcaatctgtgctttagttttagtagtcatcttattactctctcttgttagttaataaatcattttgtgaggttattctaaggggatttgaagccccttgtcaaacactttaatcgtaaattgtagtcccttctggaacacattttacatcaaatcctaaaaacttgctcacttcaacaggtgacactttattccaatctaaatgagaaagcaagaagttttcttgttttctttttgagaagtagtttgtcaccttaatgaccatacggtcaccagctttcttaattttcttgtagagctttgtatcattatcaagacaccttttcaaatcttgcaacacactgatagtgtaagtgtatgcaaatggtgcagcaacgtattgaattttaccgaacattgcctctacagcatcttcattaccttctaggaagatagttttgtttgtatcccgtgaatagcaataaccacgagaaatcttcctgttattgaacgtaaagtttctggcaataatccaacagcttgtgaggttgaggataccatccatatagctgcgtgtcatatacctgttatgattaatccaatgaacattagttaagctgttgttcaagagtttctcatctacttcaatccagtcttctggtatctttttccaatttgcttttcggaaaacatagactgtcttcccaatgacattggcaactgacttagctacatcatctggagatgagcaaaaacatttaccatcaacagtgtcacctactgccttaatataaagttggggttcttcaattgtgtctaagtcttctgacacctctttgtatgaggctgccccttctgctggaacagctttccagagttttacaacacctcttacagcttctttacgctgataatggtgttccttatcagacatcttcacaatctttaacaagcttttatcaagtttatgcagattgattaaatcgtctagctctttctcagtagagaatacaaagacaataccattgtatctgtgaaacagacttgactcgctggcatagtctcgacatgcaccacgcaagatttgattgcgtcctacagtcttctcagtaccgtttttattgcgacggtcattgataacaaacaaaaactgttcaatttggcttttacgcattgcaccaaagatattgaacatacttgcctcttgagtgtaggaaagtgatgttgcacgaatcttactctctaaagagttgaacttgacataagcaaccgggtcataaagataatctactttagggatgttgtttccgttcctatcaaccttaatattacctttaccatcacgttcatagataactgagccgtcttctgcgtagataattccacgacgaatgtttagtaattcttcttccagagaatcaagcttaacacctccccactctagctttggacacacagcattaaacatctctcgtgagttcaaacgtaactcagcataagcctgtgcagcatccatgagtgtaggttggttattaactttcttgataacatcttttgtaattgcctcagttatctttttagtagcctcaatgataacattttttgtcgtatcattcatctgcaacgcttcacgagaagctgctatagcaactgaaccaataggcatgtagatgtttacaaggtctacactcctacggaaaaattctggcaagactttgaagaaatcatcaccaagtaacgcttccatgttaactggataggcaatattacccatcaccacattaaactctgtcctgttatcgctagaacgccagctatgcttatgaatcatggcatcataaacaccttcttcacgggcgatgacattcatatctgctaatacatcgtcatacttgatattactttctggttttacagcaaagtatgagtatacgtgaccagcctcttcaaagaactttgagatacgatggtcagcaactgctacacgtacagctaaaccattaggttcttttgttgggttagtagtgagtttagttacttgaggtataccattctcaagataaacagagtacttattaacaacaccatcaacatagctagatactgtgaatgactgagcaattgcaaatggggattttgagccgatacccattgcaccaatgtagtcattagagtcattcttcgtagaagctccgtagtttagatataaactcataactttatcatgagtcaatccagttccaaaatcacgaacttcaaagtaaggctcaaaacgagtaggtaaatgcacatggaacgggatgttctcttttccagcttctttctgagcatctactgcgttacatgatagttcacgaatcactgccctttctttaaaggtatatacaccagaactcaaaaggctgaacatttcaggtgtcattgtaatctgtgcctgagacgtctctaaagaagttgaactcttaatcacttctgcgtggtcgtttaccatgcgcataatactttcctcttagtttactgttaaattacttattaaacattttaccagaaccaccacacatagggcaacaacctgcccttatgtacccctctccgttacagtagtcacattttttgtgttctgcaaagtacttacaaagaaggtaaacaaacactgctacactaccaagtgacagtaaaatttctatccaatatgcttgcatgactcacctctcatattcttgtaaaggataaagcttctgtcatgaacgatgcttgtagatactgcaaagttactttcaagcatctgtttgtttggattgtagaagctttctttaaactcgtcaagtgcaaattggtgattgtctggaagaccctttacacacacctttattgtgattgctgctgcaaccttgccagtcagttcttctttagctgctactaagattttgttgaggaacttatgaccattttcaggcttcttgatgttctcccagcgttcttctaagacaatgttgatgttcatttctttgattttcataatcttctccagaaattaaaaaggctcccgtaggagcctcttatcatatacttttaaagctgcttgtcaatattatttcgaatatcaccaagagttgtgtaaccgaactgctcagagtcactaaagactaaacgtaaagcacaagctggatgttcaagcacatctgcaaagctctggaatccatagccatctacagctttcagcttatcaccatcccacataggtgctacaccaccaaaagcagacttcttaacaccactatctgttttagggtcttttgcaagcataatctctttgccaccaatgcttgcaagagttgctttgacagcaaatgcaaaggtgtcacgagtcatgtactggtaagtgtaagaacctacaccaaacactacgttagagcttgcaaaacccatttcatacaaacgcttaaggatttcgtttgcacgttccaacgtgatagagtcaccataaataaggccgatgtgctcatccagtaccttaaagcctttagagttgatagtccctccaaagatgttatacagggttttaatagccccatcaatctctgctacaggacgagttacaacgtttgcagaaccaaccatataagcatttttcaacatcactgtatcagcaacttcaaagtcttctctgtcaacaaccatttcatagccttctgacagtagccacccagcaataccatagttgatgttttcaagcttcatgttcagaacagcattcaacattgtgtcactggcttccagcttgcttaggtgttcatagtaagcctttttagcacactctaagtggattgctttataacctgtgacaatatgtacagggtcgccagagtcaggacgaattaccagtttgccatcacgttccataatctctttacgaagtgttggtaagatttctgatacagttctccagaaattataagtatctgaaacaatacttacaataccagttgggtaagtttctgttaagaaacgacggaaggtttgtaactcacctttaaagcgtctttcttcttcaatcaactcattcccaccttcccaagcaatgtttgcacacattacagagtgttcagtggctggtacagaaccaccaatatcggaaattggataggactgtccataaatgcgtttagctgtatatacagcagggaagctatcagtccctttaaagctggttaagtggcctacagcgttaaatgcgtcatcggtaaagccggacataccacgcatagcaaagtcatggcactgataaggtaaatgcaagtcattgtcacaagtaaggtcagaccacttcttacagatacgtttgtagtgcaatgcaatagttgcaatggtacaagccttccaaatctcagcagagaaagcatcttccagatatcccgctacccaatggaaacctgaaacagtgttctggaatacaatcattggaacacgcatagggacaactgtaccttcttctacagcgtatacttcaactggtagataacctaagtcgtgaagtgcttcccaatgttctcgaccaatagcatctttacccagaacaccgttcataacttctaagatttcgtcaattgcttcttttttgtcacgttcaaagaaggtggcgttccagtggtctatcaagtaatctttaacaaaacgttgaataccaaaagctactacaccatcaattgctaaagggctgttaaaccatttatcactacgtggtgtcaggttgaacattaggtactctgttgcactaggatactgataaatatggccagacttgtaagcatccgcgtttaaacctgctggtactgcataaagtgatttagtcatctttttaatctctctcaaaatggggctttgaagcccctataaagtttcaatcaatggcaattacagtaacttgggcataatgtgttaagccacggtctttagcttcccctaaagagtttgtagtgtagatatggtcaataccattgtcaagtaagttttcaacacctttagagaaaatgccatgagttacatagagttccacacgttttgcacctgcttcacgaagatgtttagctgcttctatgaaggttcgaccaccatcacagatatcatcgagaatcatgacagttttatctgtcaaatcaacagcatcaaggattcgcataccagtaatttcaccagtcttaaggttgcgtactttagacattgtgatgtatggtttatccacctctttagcagcctcttcagtcttcttagctgcacctgcatctggagctaccaataaatcaatgcgtgggtcacttgcaaagtgtactgccacatctttttgcttcatgctctggaagcacttaaataagttttctgcaacattgctgtgagggtcaagagcacatactgcatcaaaacccattgcattaacgaggttggcaaaagctttcagtgctgctgaatcgcctttgaacatatggcggtcatatcgagcattcggtaagaacccgaaaacaacggttttcatcgctgatttatatggaacaagactgtcaatggcatccttagccaaagctaatgcaaacagtgtatctttatcatatccctgcacagtaagtataacattgtcaatcttaccagcattacgctcagtaaacttaacaaaatcttcagagaagtttccaccaatttctccagatgggaattggatgatattaaatttttctgtgtggaagaatttctcttgtgcggtgacaacggcttcaataatagttttcatagttttctctctcaatcaaaagttacgtttgttactgccattgtaagttgccctataaactgtctcgaacttcaacaggagagatattgccaaatctgtatagttgttgtcaagcacatctggcatattatttttgatatatttctcttgctcttcataggtcatcttaggagtacttaggaggttatctccttgcttctgattatcagctttagtttggataaaccagtttgtacagtgatgctcaccaccttttgtaacaggcttgtggtgacctaatgtgaaggcttgattacctgtgtatctacaaagcttattgagcactgcaatggtgttcttcacaacaaaacattcatacagattatctatggtgtaatgtgggtaatagcggaatagtgctctttcacgggtactgtcacgattccagagaatcatgtgattagagttagaagggtcgtattggtgggagtcaatgaactcttgacgttcctcaaagctaagtcttagaactgacattgcagccgaacgtttcagatttgacagatacattttatctccaaaataaaaagggaacctttacagtccccttatagtatctgtttactgagctactttcaagatgcttgtgaatgaaatctcactctcatccataaactcttttgcttccccagtaacttttacaaagttatctaggcagataatcttcttatctttcttgctgtaggttaacttaaccttttcaatatctttcttagagcttttctcttcattgaagagctttacagcctctttgaatgggaggtatgaaccgtcatcatcactaatgaatcctgtgccacagtaataaaagttatactctttagccataaactggcacagtacgaaaatctgtaatttacgagtgttataaactttgctcatgattatttagccttcttgttacgtttacggttacgagcttttttagctgcacgtttaattgctgctgcaccagttggacgatgtgcttgttttttaccaccttttccacgacctacatagatgcttttcataatctcattagccagcttttcatccatcttgaagtgctgcatgagatgagtcacgtcaacaccagtaagcatacctagtgatgccagtgctgcttttacttttgatttaaaactgttccagattcccatttcatatccttctctcaatcttttcacaaaatgttttaagttcttcttcagtgacaccaacaatactgtcaccaacgtttaaatttgttctgtagaacattgtaaagtgacctttgccaacaacttcgaatgttccagccttctcaatctctgctaaggcttccattccaacattaccgaagagtgagtttgtatactttttccactcatcaaggagttcggtcttgaccttataagtgcctaataaagaactattaaaagagataatcattatctcacctttcccgtagccaattttacgacgagatttactctgctcgccattgacaatcgccttgccatccatactcttttctccagaggttttggttatcagaaccacgatatggtttaactgttggtttagaagaatcatacttaccatcaatcaccacgtcaacatatttcataacgtctaagtggattttttcgtgtagttgaaaacctgtccaaagccatatagatttttctgggtaaacagttttaatacgtttgcatatgttggtgacctcttgaatgtttctatcgtccagaggctccccaccaagtattgacagtccactgatagcattatcattcattagctgaatgattccgtaaaggtttgcataagtaaactctttaccagcattaaacttccaagactccctgttaaagcagccttcacagtgatgtttacagccagctacgaagaggctcacacgaaccccttcaccattagctgtgtcaaatggtcgaatctccatgtaattcatctagttacctcaccaatacatataagtgctctgagcagctttctttggagacttgctgtcaatgtcaaaatcttcatctaagaaggatgtcttagaaagctgactgatttgtgttcttgtaagacgtttagtagccttcttaatctctctacaaggctgacttgtaaagtcataagagccaccttttctggacatcatatcagaatggaagtagacatctgaagcattttctctgttacgtacctcatttctccactcatagagtccgtgaacgttctttaaaccttttcttcgaattgttttactcattcaaacaccacctgacaaggaacctgtttagtaataaattcttcacagtgttctttaatgaagttttgtaatggcacacaatcttgtgcacaagtcattagcataatgtactctggattcataatattttcaatggtgtagaagacaacattgtcaaataattcatgaccgtcttcttcagaccaagttccagtgactatagctaatttgtcaagcaattcattaggcaacgctacaccatcttctacggaatcacttagaacgttgtcacctatgtacaatgtgaagaccgtatggaatgttgagtatcttacgtgattcttttcgcaagagccacactctgaggcgtaatctttaatccactctaattgttttttgttcaacttaatcataatttcaccttagttggtaatgcactgaatgctatagctaaccctaaaaatggtttgaagcatgaccataaatccatctgaccatgatatgcgaaactccattgtgggtctgaaaaaccccataccataacactaactccaaagaagaagattagaatgtggagtatgtatatcaacgtttttgatattttaaacattttaacttatctctcaactcacaataagagagtcctaaaaagaaccccagtaagacagaacctatgaacaggccaatgatactctctgccatatctaacatccaataaaaaaggtgatgtaagcctatcaagactcacaccaccttgtcaagttttacatagagaccctatcacgaatctcagcaatttttgcatcgttcattcgggattcacctttaatcttagtccacccaagatacccacacactctgttaatcacagagatatcgtgtgaatggcaaatctcacactcttcaacatcagcctttggtctgttaccacaatgttcacagattgctaagtcaaagttaagtccctgataaaaacctttcaacatccctcttgtaatacaacttttaagtgctgttaagttttctgggttagctacccttacatactggattctgccacctctacagatatggaagaatggttcttctaagtcctgcttctcaaatggtgagatgtctgctgcaacattcatatggaaactatttgtaaagtattccttatcagaaacacctttgataacgccaaacatatctctgaactgttttagttgagttccacaaagtgattctgctggagtaccatagacggcatataagaagccatcttcattcttaaactcttcagttttcatgttaatgtatgccagaacatcgtatgcaaaactacagcttccaacttcatggagtcgtttaccctcagcaagaacagacaactcatcaagagcagtaaccccaaaagaggctgtgaaagatttcacgatatcccaaccaaccttatcagtaggtttcttagtccctttgtacagaccaccttgtgtgaatgcaagagggttagaacttgctggcatattagcaatcatttcgtagcgtttcttgtggaagctgcgaatcatctctaggtacttatcaagctctttccagaaatctaaaccattctctttagaatactggtaaatcattggtaagttcaaagatacagcaccaatattagcacgaccaacgtagaactcttctccattctcgttaagatatggggataagaaagctctgcaacccatcggtgaaattaccttgccagaacgctcaaaaacctctgctacagcaccatgaccagatacgcttaagaaatctggatacattgctttagagcaacactcaatagctttactgtacagttggccttgacagatattctcatcgtgtctcttctggtcatgaatataaaccagtttagggaatacaacaggcttcttgctcttaccttgtccattcatgcgaacatcaagaatggtgtttgcaatcatgtattgtagccggttgtcttcattagacatgtctgagtcaagtaaaccaaatgttaaagttgtgaatgcaaaatcaccacggctgcaaggtacagtatttagtttcatctctagtgactggaagccttgagttaattcaatctgtagctgctccatgacatagttatggtaatgttctttaggaataccgtaagatgctgctttctcagcatgatagcgtagagacttcttagcatacggtacaagcaccttatcaatctctgctaaagtaaatccaccaaattgctgtgcagttgctgaaagaactacatcaccgataacttgtaaggctgacagtacagactttggttcacagtattcgatgccagacatttcaaagccaccttccagtactttaccaatgtcaaacaagcaacagttaataccaccaaaaatcaggtctcttaaatcgtggatatagataaaccctttttcaatggattcaagttcctctggtgttaaatggtactgtttaaagatttctttagtcagataaccacgaataattgaaccttttgtagaaattaaactgctgtcaaagttagcattttcacggtcacctaagaagagtgtgtctctggtcttttggtaaagctcatcccaattttgagcaacttctttacggtaatttctgtatgttgaataagattcataaatctcgtgattgacttctgccaaagcaccctcaacaatactgtgaatatcatttaccgagataagtaagttattctgcttagtggactttaccagaatcctcataaatgctgactcaagagcttgggtaacatctggtggaagttctttatatccaaccctgttagctgacttagttactgctgctaaaacttttttgatatccggttcttcaagtgagccattctttttaataatttgtactttgttcattattgcccctttacatgctaaaaaggtctccgaagagaccctttcattttaaatctttttagaaaacttcgaaaagagttctcttagtttttcagtgatgcctaactgtatttctaatatttcaaagataacccatagtaacatggctccaaacaatgaaccttgttcaaaatcagtaaataggttaacaacaagcataccaatcacaatagctggtgcatcaacaaccaaaccttcccaaaggcgtttaagcattattcacctctgccataaaattctgtagcgttccaacaggttttaggttttgaccgtcagttttcatgatgaatggcatctgacgaactggcatctgtgcaatatccatcaggtctgacagttcataatcctgccctaacatcctcacaacatggtcaataccacgagctttcgcaaaattctttgcagtctcacactgagggcatccagttttggagtaaattacataagtcattaaggaacctctaaaaatccactattcaaatcatctacaacagtattcaataaatacgcaccgttctgttgctcctgattagcattctgctctttatcaatttccatcttcttaatcatatacttcaaaggtggttctttaggagctataaaatctctgggaatgccaaacatatcatacagtggggcagcattatagtaaacccactcatgaagaagctttgtatttaaaccaactacagcacgtccttcggagaagatatagtacgaccatttttcttcactttcaactacttcatctaagattactttaatctctggaagaatttgttggaaagctttttgccactcatcatctcttaaagtctcttttaaaacttcaatatcaattttagtgtgaaggatttcgtcaagcataattttctggacagcttgagcaataccttgaaatttatcttgagcatcaagtgcaaaagtacatgcaaaggatgccataaaagaaatgccttcaagtgcagtcactgcaaatagaccttttagaatcactttatggaagtgcaaagggtctttgtccagaagcgagtcacggacataactaagacgatagtttatgccttcatccagtaattcttcaagaacacgattcacagtttttaatcggtcttgtacagcaatgttctgattaatctcatctaagattgtttcaggatttttaatacattgtcttacaatctctgagtaagtaagagcatgtaggttttcaatctcagactgcttcataattgcagttgcataaatgtcatcagagatgaatggtgcaaaggcaaacgctaagctcttagcaacttgggtatctgcttcccactgccacttaagaatctcaagcattacacctgacattgatgctggcacactctcaaaatcaagacgtgattgttcaaaagggaactcatcttctgaccagtcttgtgctttttgttgtttatatagctcaaagatttttgggtagtgtttgttaagtgagtcaaatgtttttctttcaccacctaaaaagattgggtgttggttaatcatagtgtgatttctcctttctttatcatctctaaggcttgttttctgtctaaaacttcccaattttctttgttaaacatgtcataaggtcttgcgtagattttaccatctgctgtagagatgtaagagacaccagcgacccatgagtcatcattttgttttatcatcatgtctgtgctactcacatagtacatcgttttccgaggtttgtggagcaagtatatgggccactcgtgatttgctattaagtctttcatagtttctccaaagttaattttagagggtcttttacgacccccatagttattaaaactaaacaccacaaccctcgcaataagcatcttgcagtgcagatttacctacaccaatgcgactgttaaggtagtacatggttttcatacctactgagttggcataaagcatgtacttcaaagcttgagccaatgataccttcttagcttttgcgtagtcaacatagaaatctgaagagatagcttgaccagtgaacttttgaacaattgcataacaatcaatcaggtcaaaggtgtcaatatcccaagcaatttcatagacatacttcaactcttcgtaatctggaacaataaacagtacgttaccagttgcagactttttagttaaaataaagtcacgaattgggtacaagccgtttgtcgtattagttgccagtgaagaactctcattaggcatgtaagcttctaatacagagttacggattccaccattttctttaatacgttgtgctaagtcatcccaatcatatttcagttttgcatcatgcttctcatcaatcttcttgttagctgtcttcggaggaacccatccttcaggatacttagtgaactgcatatactcaggcacaccacgttctttagcaagtcttaaggaagcttcgtgtaagtagtaagagtgcatttcagcaagctcatgaagcttcgtcttacctgctcttgaagagtagttcacgtagttttttgcaaggtaatgagccacatttgtaaggccaatcccaacagaacgacgcttctgaacatggttacacatcgacggatacggataatccataaggtcaataacggagtcaaccattgcaagagcataataagcaacgtcagcgtattcatcttctgaaattctccctgcaaccaaactagctaggaagcaaagagctacctcaccatcctctttcacagcatcatctctgtaaaggtctgtctctttctcaaagccgtacactggcaatacaatttccatacaaagatttgacatcttcaaaggttctttaaatggtgtatgtgtgtttgcattatttgtgaagaatgggtacacacggcctgttgcataacgctgctggataaacagtttagcaatttcacgagcctttactcgtctgtgcttaacacctgagtgtactgcatgaccaactgccatagcaaactcatcagcagatgctgtgtagaacatgtcatagagctttggtgcatccttgtaagagaataacaaccagtctgtatcatattgaacacactgccagaagtaatcatttgtaccaaatgagtagtccatctcgttaatacgtttcgaaggaaccgttgtagggtgcttcaaacgcagtaaatcttcaatctgtgggtctagagcagtgtagaagttgttagctgaaccaccacggctcttctgtttgtttgcctctacagatgaacgtacaagcttgtaataaggcagtttacccatgtgctcaatagtattttgacggataccatcaccgatagtgcgagtctccatcaacataccaataccagcttgctttgtagtcatatcataggcaacctttgcagcaataccgagtgactcagcagtatcattcgccttaatcaagcagcatgacgcataacctgatttagtggctcttaaaccattcagataaggcgtaggagcgttaatcttcaggtcagataggtaagtgtacagcttaatcacatcttgcagcctacggtgctttggttgcttctcaaaggctttcatagccatacccataaacataaattgtggtgactcaaaaagtcttccagttttaatatcacggataccatacttatctctaaactgtttcaggactgcataaccgtaagagacatcttttgagtggacgatatacccttgtaggtattcaagctcttcctgtgaatagtccatcttctcccaaagtcctgctctctccatatttttaacgaaggtaatcagtgtaggaaccttagtaaagcctccaaaggcttctttgtagataatccctagaagtagccgtccagccatatctgagtgctcttgagtttgtttatcaacacaaacatcaatcatggcttggtgcatctcttttgtagtgcaaccctcataaacacgtttcaaggcttccatagtgacttctgaccagataatcccatgcttatctgcccatgatgcccacttattcggtctttctgggtcaaagcttactactgtaccgtttgattttctaattgtcttaatcatttttgaatcctacagatgaaaagagcctccgaagaggctccctagtttaaatcttgaaaatttgctcattctgccacatatcgtaaaacttatcacttacctttgtgactgaagaagtgtgcatacaattcaggaaccatgatggtgtattagcatccataattgtttcaagcttttgtgtaacgttcacatccatgtctgcaacaacgtaattacctcgcatcttgcaaacaattctcccaaacaagattgcacttctacctcttcggtcatcacagtacataactgtgtctccgtgcttaacgtcttgtccgatactgtccacacctaacttagcacctgagatgatgtcatcataagatagcttttttgctttagacacgatatttctccaagtttctttttagatataaccaaccgtagtcagtcctttctacacccataaagagtggagccatcacaactttaccaaaagtatttctgtgttggcagatttcaaaggaatactctttctcgatatcaatcccatatttgtacaggactgcttttaaaacttgttcattatccagaagtgtatctggagtctcaccatacttctcaagtaacgggtcatacataaacaccgtcatagaaatattatagtttgagaagtggctcttctgagccacgccttctttattaatgttctgtgaatccattataaccaaccccttttaaccagcacttcaggttatcagcatcataattcagtttcgggtatgaggttacatggaactgttcaccatcgtagtagacaatatctgcaatccatagtccattctctttacagaaatctttatctgctttagaggtaattggattaacttctacaaaaccatctacacccaaatcctttaagctacctacaatacctttgcagataacgcatgtctcagatactaccacaaaaagttcttggctttcaagttctacaatgtattcaccaagctgtactttaggtagatttgattgcttcataatcttcccatcggctgcacgaacaatcgcataccactctttaccatcaactactgacattctgataatacactcttgaccagtacgcttctcaatatctgcaagacgttttaaagcttcttcgtagtcatctgtgtacttcaggtcattattatggcaaacagctttcatagcaccgttatggtcatgttgtgacaggtaaatcaaaccgtcaaggacatattgcaaatctgcctgagcatccagtgtctcaatggggtctttctcttcaattgcttttataagctcttttgcttcttcaagcatacataaagattgagactttaaagattcccaatactcgtcactgtaaggttgcttttgagtgtttccacaacgaaggttccagttttttactgattctcttaagttaaacattcggactccttaaaatatttgttaacctttagctctcgcgtttcaatatattccagatgctcacgtagctcttgacgtttaagcattaggtgctgcatttgagactcaacagcttcaacttctttgattattgactctctggcatttgcaagaatgcgctcaagttcttcaatcttgctgtcaacttttgctacatgacttacagctttacttttgttaaacattttattctctctcaatagttatcaagcacaatttgctttcattaccttatgtattcttttttcagcatcttcataagtttctttagtaataaacttaattgctgcaatattggcattgtagaagagtctgagtttagagtcaatcctttttgtcattacatcgaatttatgttgaaggtttgcttcaccataaactagaccacctttcgtgtagtaggtctggataatgtaaaagtcaaaaaattcttttccaaagctctcgatatcttttttaatatactcagaagaagtctcataagtcatccaatcactctccttagtgactaccttcttccgagtcttgccagcaacttttcttttggtcacactattaagctgtttctttccgatataatattgtccggttttcttacagtgtactaaatagacaaaaccaaaatgtttagtggggtcaacttccccacataaagatacccaatgaccgtaggtaggacaattgccaaatcctttaatcttcattcataagcttctccgtgatttgaaaactctttatgtagttcttctcttttctttgtgactgcatctttggcatcttcaatactgtcaaaataccccagaaagtgagataccccgtgtgccataactctggccttccatttttggttagccttatagaaagatacacccttaactccagatgtgttgttggagtgtgttcttctattccaagagttttcagcttgtgtgcaaacccttagatttaatctacgattatcattcgtaataccgttgcgatggtctactctgttaggtaggaaaccgtctgtgtacaggattgctaaatcatgagccatatagaattttcttttaatttttatcctaatataacctcgcttatgtggtgtacctgcaatggaacctataactgttctgttagctgatttctttatccacgtaaagacccctgtttcggggtcatagtgtagatattctttaagtgtctgctgagtcaacatttggagtccaaccatacttttcaaatgaaaaaatatctgatttatttcgctcttgataagctaggaagaaatgttgttccattaactctaatggtgtctttgtaacagtctgcccgtcccatgatacataggtgtaagactcttttttagcatagagttcatagatagcatcaagacactctttgtaggtctctttaccatctaaagcattcatcacagctactttgccagcacctttaagtccgaagtagttgtctgcattatccccagcaacagcctgataacacaagaatttaaaacctacaccaactgtttttgcagcttttggttttgatttgatagggcaatcccagatatcaccaacattattgtcagcaataaaaattagtggtgatttttcataagtcatatcaatacaataagtcccttcagcttgtcgaaggtctttgtcaatactcataagagcagctttcttacccatcttttcagctttagcaattacgattgaatcagcttcgaaacctccctttaagagcttgaactctggtcttgacagaagatattcacgacaagcgactaagtgtgttggtgtgacagcatctttacggttaccttgatattggtgctcaagacctttaatgtctttatgtttatgtacacccttctctgtcaagtaacctacccaagttctttctttaccaacaaccttaagccattcctgaagaacctgctgagtagccatgatagcttctttttcactcttagcttctttccaagtctgtctttcccattcatcttcatcaaatgctaggccaagctcttctacaaagattctctggtctgctaaccatcttgcagcgtcttttgcattatcaaatggttcagattcttctgctgtgagtttattgacatatttatattttgctttctcaactacacaagcacctttataggcaatactgtcagagtcgataaagacatgtgtaactgaatcggggagttttgttaacgtatacttctccattgtgactctccatatgaaaaagccccatactaagtacagggctttaaagtaacttttagagattagtcttctgtatcgaagtcttcatcttcttcgtcatctgggtctggcaagtcttcatcgtcaccatcatcagaatcattcgaaggtttgtggtcttttgcgtcttcttcagtgatttcaccgttatcttcaacaccatcaagaccaagcatagccagttcgtcttcatccagttcaggttcgccattagcaccattaccaccagtgtaaggcacaagagtatcaatgataaactgttcctgaataggttttgtcagaacattgttctcaaaagtgtagaagtgagtagaaagaatcacactaccaaaagaaccgttaccgactgcaatatctggatgaattacatcatagttcttgtcatcttcatgtttgtcagatgcctgagctttgattttcttcatcggctgtttaacagctacacgcttaccatttacttcttcaatcagcattacagggaatgactgtttagctgtccacacagcaccatctttataagctgctgcacgacttactttcaagatgtggtaagtgtctgcttcaaatggtggtttgcaaccaaacttctcttcgaagtcatctgcatcaactgcttcagtagtaactttatcccaaccttctgggtttttcttagacttagtaaactctttaaacagcttgttaccatcctctgccagaattgaaacactgtagttacaatcttttcctgggaatttcttatcaatggacttaccttttgccggacgtggtgaagtgttcaggtaataaaaccaaacatctttcagcaggtaacgcagagtttgacgttcagtaccattgtacttctctaccggagctttcattttaacaactttagacattatttaaacctctatctcaatttatgaagagtaccaattctaatttgttgatactctattgtcaaacattaattactggaagttgtgcttatctttagaagctgctttacgtgcagtttttccagttcttacacgcttctcttcatagtgttttgctggtttaccagcagtggcttttaaggaatccccaaaaacttgttcaaatgctttagagtgtttcatgatattctccgatatacttgatattcaaacacattttctttaattataaacagccaaccaatattatgtcgaggtatagtaatagtgactgttattatactgtgatggaacttttcttctacctcaacgcctccatcacatttcatgtagagcattctatagaacctatcaacctttgtcaaacactcttcctgaaatttatttaaacttttttcttcaaagaagggtatgaacccctctcttttataactatcactcagtttcgaatgggcagtcatctgcatcctcacttaatacaggtggaatcgttgagctttgacgctgttcttcagtataaacttcaccagtctcacggtcaaacacttcatcctcataatgaggtaaagagtcatcataatggtcttcagcttcaccaataccaaactgttgacgaatgttttctgctgcaccatcaatgtcaacaccacacccagaagctttgataagccgtcctgtatctggattgtaccaagtatgaccagcaatacctgttgacttaccatgacgacgacacttagttaacttgatttttgtcaagtttttcttaacagggtctgggtcaaccttgttacgcattaacagaatgttgttcatggaaatctggaagtatgcaccagaacccttaatatcctcttcagagatatctccaccttcagagttagctttctgaccacctgcactcttacgaacgtgacagacgttaacctgcgcatactggtagcgtttgcaacgacgcaatagttcagacaaaacttcctcttcatccgtatctgaacgtgacagagccaacgtaatagggtcaagaataataatcttacagtctaaactgttaacaagatagtcaacaaattccagcaggttatcttggtcaattgccccttgatggtcaacgatatggatacgacgacccttagatagttctgcgtgtgctccctttagttcatcccaatcacgttcgtcataaggaatctcagaaatctgcttgctcaggtggattgcacagagcatctccatcaactcttcataggtatcttcaactggaattacaccaatattataatcagtttctttccaagctgaataaatcatctcacgggtgtaagctgacttacctactgaagatggtgctgccagagttgtaatctcacctaaaccataaccaccataagtcaatctgttcaaatccccgaaagattctgggaaaggaatcaatggaatctgaccacgattcttcattgcctcaaaaccatctgcgaagttcttgataccagcagggcagtaacgaggtgcattgtagatacgctgtttaaatccttccagaactgtatctttctctttatagaactttgtccaccattcgttaaggtcttttacgccttctggatactggaataaacgaaccttctcaataggaaggataccagcagcctctttggtagctttagcacctgcttcatcgttatcaaagcacaagtaaatctcatcaaatgatgtaatgtactgatagttgtctttgatagacttaatgtttgcacctgatggaacagatacgtgacagtaattcttacgacgagacttatctttaattgccagagaagtcatgtagattgctgtagcacattccatctctccttcccagatgaatagacggttgccaccttctggagcaatccatgaaccgaacattgccagttcacctttaatatctccaacaccacctgaaaagtcttttaacttaccacgtaggtgttcttttggatggtcttctgggtaacggtgacggacacggtagccaacatgttctagtttgccatcctcattgcgtttgtaagttgggtagaaatgtgcgtcaatttcaccgtcactgtcaatgtcaaccttgatacctagacgttcaagaacttttgcaggaatcttcctgtctttcaagtccattgcttcaaggttttcttttacgtcatctaaatccattccacggaaagtacggtttttattatctgaaccagtagaataagtgctcacgatttgtcctttatcaaaatcccactctgggaaacctttattacaactaaagcaagtcatcgaataagaatcatcatcatgatggtagattgaacccgcatctgatgagccacaacgtggacatgcacaatgaccaataaactgaccagcctctttcaatttacgacctttagacattagcacctcttcgttgtagttctgccttcaatccattttcaatcttgtccagttcatgaatttcatctgcaatctctttccttcgtgattcaactctcttaaggcgttcaatcattacctcattggaaagagatgagagttccactaaacgatggtcaataactttgaaattatcttttactctcatcttcattctctcttttaaattttcaggatagctcttagcttgctttcaaggtctgcaagagtaccattattatgaataatgtcacgctcatatttggtggaaataccattttctgaaacgtgtgatgaaatcttatccgcattgtctctttttacttcaatagtttggtgtgcaaatctgctcagccactcagcttcagagtcaaatcttaagtcactgattaaaacaacaccttcttcatttctaagtgagcaaacatcaaagaatctaaccattcgtttctcaaggtctttagaccagaacctgtcgcccatgactttacggacaacttcagttccccaaatctgttggatttgtcttgaagagaatttatacttctcactgagtcctagacgcgttaatagagtcggtttagcgaccttcttaagttccatgattaatcgtccagtcaactctgacaagagtttaaagtccatatgataacgttcatctttgaaggtgaactccatagcttccgtaactttaggcatgagtttatagtatgataagtcaaagacttgtggtgtctctttagtttcaccgtacaggtcattccaagttaggtcaaatatctttgatgcagagagtttaagattgtctgcaaaagccatcaccgcaacgttgtaaccgtactcatcttctaagatattcttcacaatagagcatgaggtgtcttttccagaacgtgcctttccaataaatgctataatactactcattcttcctcccttaattactaaaaagcccccaactaaggaggcttgtaagatttataaagacttagtgcagttgttcactagtaatctgtgagtgctctttttcagccatagctgagacttgcttcacaaaatcatcaccaaaattaatacgtagcttctcttcaatgattgaagcaccgatgttaaccaaaacatcattgattgcttgcatactgacaccaccagtcatctcctcaatgaactcaatgctcgctcccaacatctgagaaagttggattagtgcaacgatattcatcattgtagaaatagctggaatcatcagagcgaccttcacctgtagcggttctacattagctacagaatcatctttcacagcttcttctaaggactctttagtttgctggataatctctttgattcgtggggttaactcttctacaccccaatcaagcttttcatattgtgacagcttctcttccatgcgcttctcaagagcagccataacagcactcgttgatgcaatgattgtgtcaataagggaatcagtatcctgagtcagcaacagttctttattttcgtctgacatcctgatatagtcatttttttgcatttcataaaaaatgaactcagcaagtgcatcaacaccaaccgcaatagatgccagagctagtgcatggtctgtcaacatcatgttaacctgttctgcaaaagcaccgttatcagacttatctttaggtaagttgtaactctcaggaataaactgttctgtatagttgtttccacgaagaatcatagccatgctttctacggcattgattaagaatgcttcgtcaatacctttttctttaagcatttggtctacagtagtattcatattatttctctctcagttggttaagtttctttctagaacattttaagttttcagatgaagaaaagtcaagcgtcttttcagaacgtggatagtaatgtctatcccaagaagtttcaacatcgttaatcagtgatgctagactacacaggtctgtaccactttgcaacttctttttaagagaatctagtaaagcaaccgtatctttagctttacgcctcgctgtagcaacatctttcatatgctcaaatacagagcattttaagtcatcttcatagttttcagacaactctatttcatgttgtatatcaaccattctcctgtgatggtgagcatattcccgttgagcagcaatgtcaagttcttccaacttctgcaaagctctaacaaagtcagctatgtctgggtgaacaaacattttctgcatgtaagcctcttttaagtaactattaagttttaatcttttatcttaacgtattctatacgttatactttaaagctttttaaaagctattaaataatcttttaagagttttaaagtgtctgtatagttaaactgttaagtaaccttttaagtaaaaacccacttcgcaaagtttaacaccttgtcaagtacattgtcaacttgctttttataattttttgcagtaatgtatgaggttaatgatttagagggagtagaaatggaaaacgtagattttaaaaacttacacttagttggtgatacagaaactgatggtttactccttgagttcactaaagtacacgtcatggctttcgcagactataaatctgacgatgaagagccacctgtatgggtctttacagatgagcctatcctcggtcacaaatataccaagtacattaaaggtggtttgcgtgaaggtatcgagtttgcgttaaaggcaaaacggctttgcatccataatggtctggggtatgactggtgggttttcaatcacattgcacctgatttgtggaactttgataatccaaagtgtaagccgtggagtaatttctttcaggattctcttatccagtctcgtgttcagtggatggatagaccaactccaaaaggctataaaggtgctcatggcttggcagcatggggtgctcgtgttggtgtacgtaaaccagagattgaacattggggtgtgtggaatgcagaaatattcactcgtgttgtagaagatatccgtattaacgccaaaactaaacgcgcactggataatgaatatctcaagttaaagaagtgtggcatagacacttatgaaacctatatgcgagctaaagagacatctttctggatgagtcaacaggctatcaatggttggaaagctgataaagagcttatggagttccatgtaaaggaacttgataagttgactaatgagcttgcttcagaagttgaaccacatcttcctccaactattaagaccaaaggtaaagtcactggagaagagtttgcaaaagcttggaatgagtatgttgaaacattcggtcatgcagatggactgaagagaattaccaagtaccctaagacaaagtatcgtcagcaggtacgtaacggtgagatgcagacatatgaaatcaagccatttggtaagccaactacaaagatttttaacattgaaaagcggaattgctatacaccaaccaacactgtaactggcgaagagtacaaggaaggttttgtagcaatgaaggatgctcgtgcaatttgcaacgagttgaatgcaaagattggtaagaaatgcaaagactggaagccagtaaaaacagttaaaactgtgaagtactataacagtcatgttgttaaccactttggacttgagtcaagtcgctacacaggtttgattgatgcaccatatacaccaattgagttcgaagtttctcgtatgactcaggtagcagttgttaaagactacttgaaatcagttggttggattccagatgactggaactataagaaagactctgatggtcgtcctgttaaggtttgtcgtttcaaagacaacaaaaagatgattaccaaacatcctaaatgggatgagatggttgagcgttgtggattgagctatgttgaacatgaaggtgtccagtacattgagcataactggtctgttaagaagtacacagacttacttgaaccttgtctgattaggacttcaccaaaacttactgaatcatcttatgatacgattgaaggtgagcttggacagaagattgccaagtactatactttgatgcaccgacgcagaactattgagaactcaaaggatgatgaaaaaggttggttgaaccagattcgtccagatggtcgccttagtgctggtgcaatggtgtttggtacttcaactggacgtatgacacaatatggtattgtaaacgtaccgtctggtgctgctgtctatggagcacctatgagggaggtgtggacttgtgaagaaggaactaatattgtttcagcggatatgaatagcgcgcaacttgtactgctgtgtaactttatgggtgataaagatttcactaaagcggtgacagagggtaaagaagaggttgagtttgtcaaacaagaggatggtagatattattgcaaacattttgaccagtatctaaacccagctattgacaagtatttacactatgattcggaaaatgacttatacgtagtgtactcagggactgatgcacatactttaaatagtatctacttcagtcttaataaagaggaagacatcttgacatgtcgagctacacaggatgcaagtcttcttcaagaaattagtaaaggtcgtaagaaagccaaaaatggcatttatgcgtcagtggcgcatgtaaaacgttgtgaattgctggaagcctaagtctgtaaagatatggtaatcagcagcgaagtctctgttaggactcctaaaacaaggagagagacaatggcaggttacgaagagagattatgcactgagtgtggtacggagtttaaaccataccaatacaatcagaaacggtgcagaagttgtgtattaaagtttgacagaagggcagaacacagggttcttactaagaaatgtcctagttgtgagaaggtgttcatgccgagaacagcaaggcaagtttattgttcgtcacattgcggtgagaaaggtagagttaagaattactacctaagaacatatggattggaaagagaagaggttgaggctctactccgcaactgtgattacaaatgtcaaatttgtggcggtaaaggttttatcatgaatggttcacgacacaaggctaaacttgttgttgaccatgaccataaaaccggaaaggtaagaggtatgttatgccacaattgcaacagagctttggggttgcttgcagatagtgttgataacatgcaaagagcaattgactatctaagcaaatgatataactatagagattcctaacagagaaacgttcagagactatcgaaaagcatacttaacgtatgaactgagtagagtagggtggtaagctaatgaccatccccaagtgcaacggtagcattactgctacgtgatatagtccgacactccgtagaaatgcggagaagttcataagagaactggtacaccttgcgagtgtattgaccagatgtattgttcggggctggcgatgagaaatttgctaagacgattaaagcatctacaactgaggaaggtgctttaactaagcaaacatactttgtacgtctccctaaaattaagaaattgatagataatttagaagcagacttcaaagccactaagaaggctcttgaagaggtatttggtaagactgccgcaatctctaaaggggggtttgtaaaggttgctggagcttggttgtggtgtaaatctccacataagttattgaattatttgcttatgggttcggaaagtcaggttcagaatgaagccatcaacctcgcttgtagaagaacttgtgaagagggtcttacaaagttgaatggtcgaaaaccagctattggcgctagattgcttttagcatatcacgatgagaatagctgggagtgtccagaagacatgacgcaggatgttaaagcagtaattgactggatgtatggtcaagcgtcaagaaatttagggcttaaggaggaaacgcttgtaactggtactggtaaagttggtaaaaattggcttgaagtacactgattagtattgacaaggtgctcatggaagagtaccttatacaatatcttatataggaggtcacatgacgctttcagaaattgaagttcagttgcaatgtgcaggttattctcaaaaagagattgaagggatgtttattgttctttgcagtagctgtaggaatttgaaggtaaatcctgaagttctacgtagagtagatatctttattgctggaggcaaaatcgcaaaagataaaacaggctacttaaaaggtgagcgtgtaaacattgagactttactatgagccaccgtggaagaacccatgcagcaatgatgaaaggtgcttcaaaggaaactatcaagaaccgtaaacagaagttgtttgagaggatgaacaggttaattgacaattcatctctgtcaggttctgagaaggttttcttgaaaggtaatctgaaaagcattgcacaagaacttattgacattgagtattggagacatcaaaaatgacacttgcagacgttattcagcaacttcacaacaactgctacactccagagttgattcaggagatgtccattgtagttatgcctagtaagttcgtaaaaggttttaatcatgaggctttaaaggtcgcgcatattcttatcgttgatggtaagattgcaaaagattgcacaggagtactcaagggtgaacgtattgatattttggaattgttatgaataagttatatagcctctggggaaggatgggtaaagactctgattggaacctgctaaaaacaaacgtaaaaagtaatgaggtagcacactacaaaacacattactacaaaactttcagagagcttgagtatcgtgaccaacagacaggagaagtattatgtggaaatcagtaattgtacttggtgttatttttctagctggctgcaacccttcttatgaagataaaaatgcttcttatagcctcccaccagagatgcaagattgcaaagtctataagttacatggtgatgccataagcagagatattgttgttgtcagatgtccaaactctcaaacaacaacatcttatagctatggcaaaaatggtcaatcacacactactgttattgagtgaggtgttcaagatggaagtcctagtaaactatatatactgttatgatgttgttcactccactacaaccgtagctcaacgtaacccaatcgtcccacgagaaggtgagttggttagaatcgagggttggacttacactgtggaaagcatcattcacaagtttgatgttgctggtgatgttcaagtcattgacgtagagattggtggtaagagaaaatgaaacctaagcagacaatgacagatgcaaccttcacagtacacagtttcgatagattgcacatcaaagacacaaaagatacaccaaaatgcgttgtacattttattaaaaggtatagcaatatgtcctatatgaaatctgacaaaggcaatagcacaaatgaataaagaagacaagcataaaaatgctgtacggactcctgatggaaaggttcaatccttcaccctgacagttggtggaaagccatttcgttgccactgcggagcaaactgttttcataaaccagataaaaatgacttagagctttacgcatgtaatgcttgcaacacttggtatcactcagaggaataaaatgacaattctgtacaaacaaaataaagatggctcttttaacgtctggtcatgcgttgctgtaggtgacaaagttattacaacctacggtaaagaaaatggcaagatgatgtttgaagagtatacagcagaacctaaaaacatcggtaaaaagaatgagcgtaacgctgaacagcaagctctctttgaagttgctgcaaagtataaaaagcaagttgaccgcaaaggttatgcttacacaaaagagtctgcacagaatactgagaaggtaggtgtacagcttgctcatgatgctgcaaaggttagtcatgcaaagtatctgaagttccctgctgatgctcaaccgaaacttgatggtgtccgttgtagaatttcaagacacttaagtattggcagtgtatttatagcatactctcgtgagaatactgtttataaaatccctactgagctaatacctgacattatggcaatgttaaggctccacaaagatgttgatgaatttgatggagagatttatgctcatgggtgggacttagaagatatcgtctccatgattaaaaatgaagataatcctgacagacacttacttaagttttactggtatgatatctgtgacagtactaagacgtggcctgagcgtagagatatcattgagacttcaccattgaatgactttagagatggctgtaaagttgtccctgttaaatctcgtcgtgtaaattcttgggaagagtttgatgaagctcatgataagtgggttgaagctaagtttgaaggtgcaatgtaccgttcaatctctgaagactccttctatgagtgttgtcaccgttcatacttcttgattaagcacaagaagatgcacactgaagagtttaaagtgactggtgtcaagaccgataaacgtggtcatggtaagttcgttgtagaaactcttcctaacgtctttgtagatgtctcatggaagactactcatgagaaaaaacagtaccttgctgagcatcctgaagagtttatcgggaaacctttaacggttcagttccagaagatgactcgtaaggggtctttacagttccctgtcgggttagttattcgagactacgaataaaaagttgttgacatagaaaatttggtgagtatactgagcagcataaaccaacggatactcaccagcatcacttaagagtcttctaagaggatttttaagcgatgctcggtcgggtttggtcgggtttggtatggtatggtgtggtaaggtagggtttggtatggaaggatggcagtagccttataaaaaatctgccaaataagctataaaatctgttgacaagtcaggtgttatagcttatttttatactcaatgtaatacaaaatttaatataactttgaggtcattatgaaaaagattttattagctgctgcaatggttatggcaatgaatttaccagtcaatgcaacagaacttcccaatgtggacttatcaggtgttccagaagacacttgccagattgttaaagggattgctttggctaatggtgagttacttaaaccaatctctgaagagtctttaacagagatgactgacaaggtaactgactatcagtatcgtgttcttgcagagtatttcctacaatctgcaaacattaaagagaagcaccatgatgatattgatgtgcaggctatgctcaatcatcgtattcagtttaaagaagatttgatgcaaaaagctatgtatggtgttgagtatttcttagaaaacagaagctgcacaggtatttgatatggctcttaaaaagttacatcccagaagtggttatggtaagataattgacgatacagacggctttacagtctttacagttatctgtcaagatgattcacagattgaaaaggctcttgatgattatcttaacgatgaacgtgaaaaggttagagctacaaacatagactcattgattgacacttcacgcaaacggaagaagaaagatgaatgaggtttttgacccttatgctccacaagatgattgggaggctgacagagaggctgaaatggagagttatatttgtccaatggatgtagacgaaatgagagacttcgttgcacatcgttttaagagagagattaaatccagaggtctttctcaagagcaagttgctaaaatttgtggtatctctcaagctcgtgtatccaacataatcaacctcactggtaatgtctctcttgagtttatgttgggagtatgtgaaaaatttggtgttaattttaatttaaggttggtagattaatatgaaacgtgaaaacattatccactctgaaaacttcgcattaggcttttatggtgtacctactcaccttgaaaagtattacggtgtaaagattctttccaatctcattatggcttacaaagatggcaagattaagcatactgagaagaaacgtgtcatgggttatatggctgtaggttcagcaatctctaacattaagctggaaaccactagcagtcagattgtgaaagaccacttcatcaaagagctttaccacaaccttgatggtgtagatgttcaggctgtttggctggatgttgatggccacaattacacaagtttcgttttcaaaaacgatgacattaagtgtctgttcccataataggtgattaactatgattgatatcttcttacaagatgcccattcagatttccttaaagtgatgcttaaaaagtttatggcttcacagtataagaatgaagcatcttttaaaatagttacatgtggcgatgaagctggttttgttgagattgaacatgaaggtactggaaagactgtttgcaagttacctgatagtatgttctctaatacgttcttaacaaagaccagtataagagttaagcttgttcctcagattgaaacatactctggtacagattaccctaaaggttttaagtcactgatgaagtacttcttagatgactttgtgagtaatcttctgagggaggtaaaagagagtcgcacagtattaactgtagagaatatgggaggcactataagggttacttctgactgttttgttatgagcctcttcgacttcgttcctaagaactttgatggtattctggatgaagaagatgactgtgtagactttatattggttcttgaaccagtttttgaggttaaataaatgaaaattgaacactgctatgaacctgacggaacacctacccgatgcccacattgtgggtgtacagatttacaatgtgaagttcatgttattgtgaacggtcatatagctgaagagagcactacatgtgttggttgtggtgagcatctatcattctgggcttatggttcataccaacctacaccacacttaatctaccatagcaataaagctgtgaagaatgttatcaactggttcattaagaaaggatttacaaaatgattaaattaatctttgcaagtggtgaaaatggcgagtttggtactccaactggtatgccgtggcctcgacataaacaggacatgcaagagtttaagagactcactaaaaataacttagtagttatgggtaacgagactttcaagactctgggtagtaagcctttaccagagcgtgcaaacctcatcttaacaaattctgtaccatacttaggcatagactttggcaaagatgatgtaatgtatgcaaaggccagtaaagagtcatttggagcatttttgaagtatcttgatagctctattgatgaagatgtctttgtaattggtggtgcaggtgtccttgtaaatgctttaccgtatgctgatgaagtgttccatacagttttccataaagttactgaagaggtcactgtgcatttaccttttgaaaacttctttgagaagttgtatgataaccgtgtatttacaaaggtacagtcaaaaccatcagaggatggtaaagcaacttttgaaatttatgttccacaagtaaaaggacacttttgatatgtcacaagcagattcaagttacaaaaatatactgagccatgttttatcagtcggtgaactgcgtactacacgaactggagatgttatctctgcatttgctccaccacagtttcgctttgatatgcgaactggtttcccgctcttaacatctaaacaggtgtttacacggcaagttattggggaagctttatggttcctgaatggtgagaataagctgggtgaactccgttaccgcacttggggtgaaaatgatggagaacgctggactatctggtcagatgattttaaacgttggttaagctctaattattcttctgaacaggattggttagacgatgcaggtggaagaatctacggagttcagtggagaaactttgaaggtcataatggttgtgttgtagaccaactagagaccttagtaacaaagatgaagggtgatatcacagaccgttacatgcttgttaatgcttggaatgcagcagatattgcagcaaactcaatggctttagcaccttgtcatgttctgtttcagatttatattactaatgagggtgaagttgacttacaatggtatcagcgttcagtagacacctttttagggcttccatttaacattgcttcttatggttttattctggaagttctttgtaagatgactggatacactccacggtacttgataggtgtctttggagatactcagatttatcagaatcatatgaagcaggtttatgaactgatgaacaatgaagagttccatgcaccaacttttgagattggtatcccacttaacactttaagtgacttaaaacacctcactgcaagtgactttatcggtggtattaacaattaccaacatgcaggaaagattgaagcacctttgtcagtaggtaaataaaacaaaaaaggctcccgtaaaaaggagccttaaatcttacttttcagtattctttgtgttcttttcagtaatagcttggagggctgatactgattgagccagattgttcacactatcagagaatttatcaagagtcttagtaagttttgcgttctcacccttaacattctctaactggactttctggttctccatccctagctgaagcagcctcatatcagactgtaaatcacgaatagctgaataattacttttggaataattatctaactgctgtaactttgttgtgatagacacttcttgcttaccatccgagacttgcatggtggtatatatcccaataacactaaaaatactaactacaattgcaccaatattatttttaaaagcttcctccagccacttcatttattctcccccttaaaagctttttctaagttatctacgaactcatcatcaataggtgtgtctgttttactcgcaaggtatcttgcaagtctaaagaacactttctcaatcatgtattcacttagaagggataaaatgagtttccagaagaagctacctagattttttagaagaattgctaggattgtaggcatttaatcacctcatcagccaagatggtgagaatacccataaagaatattaacaccatcttaacaatcagtcaacaagggattaagcagttctcacccaagccattaacttgtagaactggttagtaacactaaatgctgaaccagaacctgtaccaccagtgttaccagatactttgtgggtatgagcaccgatacctactgtatgagagtgagcgccagcagatgcagcagtacctgatacagtgtgagagtgagcgccagcagagcttgacacttggtttgttcctgatacctgaacacgctgtttaccaccgatagagtcaccaccgtaacgtccacctacagtgtgtgtatgagcaccagtgttgttagtagaaccactcacagtgtgtgtatgagcaccagtactgttagatgtcttcgtaccatagtcaaagcttgacgttgtagctgagaagctgtgggtatgtgaaggtaagttaccaacagataactttacagaatctgaaccgccagttgtagcaacatctgaaccgtttgctgctgcaattctgatagttctaccaacaccattgttcaggtacgtccaagttaatccaggaagtgcagtattagggtcaacattactgttaaaccacgttacaatacctactggatagattttattcaggtctgtagagtcactaattgcctgcgcaatcttctggtcagtttctgctttagtgtatgcaccaatatctgaaggggttggcttatcttggtctgtatagattttagcccaagtattctcaaatccatacccatctcttgctgacctataccaaacaccaccatttctatagttaaattttaattgagcagatggtgtagagctactaccctgatacatatggtagaccagttgcgttgaaccaccagatttccctgtaacattatacagaccagttttagcattccaagcaacaccatcactatcacctacctctgtaccagtaccagaagaggaagaaagcatatacctagagtttgcggcagattgcgtataatatctggaatctaagttagagaaatctgaaggttgaacctgtccagtgattgctaaagtggcattcataccaaatgatgaaccaatggtaagtcccttgcctgtcttgtagttatacaagtttaaggtgtcactggtatcaccattaccaatataccatttattagttccgtcaggagcaagacctctaatgaacaatgaagttgtagtattttttgctcttagcgcaagagcttcaccaccactagagaggataatctgtttttcagagaatgtgttagttgcattagttcttgcaattgtacttaatgttgctgccggaatatacctagagtcaatgttagagaaatctgagggttgaacttgtccagtgatttgcacggttttattgataccaatagttgtacctagtgaaacctgcgaacctgtaaggtagttataaatgtttagacgtgtatcttcagaatctgtaccaacataccatttatttgttccatcagacttcttaccgcgaatgtaaagtggtgtacttgcatcagtattctgcaaagtaatagcattagcattcttcttaacaacaaggttagtgaaagtattagcacctgtaaaagtgttatccccagctaactttgcaaatctctgattagctgctgtctgagtaaaatatctggcatctaagttagagaagtcagaaggctgaacttgaccagtgattcttagtgtcttgttaagcgatgctgttgcatcaagtgtcaacattgagccagtggtatgattgtacaaataaacattagtattgtttgcatttccttgtccaacataccaagtttgctggttattagcatccctgccttgaatatatgaagcacctgcattggttgctttcttaagggttaactgctcaccatcagttaaaattgtttgcctaatgctgaagttatttgtaacagcaagccttgcatatcttgcatcattctcttcattagtcctcatcccaagttcattagggcttggtttattcagagtatggtaaacagttgctgaccttgaagcatctgcaatagttagtttaagaccatttgcgttgattttaaaagtctttaaaacatccccaatattaatctcagaagagcctgttgggtcaaagactgccttaccaccataaagtaacttgacaatccctaagtcacccgtcatagtactaccagcaatctgaacaaatctttcaagcttaaaagatgctaagaaatcttgataggtcattcgacggtcttcatcacctaacatttcaggtctctttttaactctgacatgaagaaggtcatctgaacggattgtgtcgattgagtttaattcactcaacttgtaatctgccattattttgtcctctagaaagagggctatacaaccctctgtaaagaatttttaagcggttctttgccatacataaactacaaaggatggctgttcaacactaaatgcttgaccagcaccagtagcacctgtagtacctgagtgagtatgttcagatgactgcaaagttactgtacctttgtgtgtatgagcaccaatcccaactgtatgactgtgagcaccgattgcaacactgtggtagtggttaccaactgtattagttgagccactaatagtgtgggagtggtcaccagcaacacttgaaatctggtttgttcctgatacctgaacacgctgtttaccaccgatagagtcaccaccgtaacgtccacctacactatgctggtggttaccagtggtattggtagaaccactgactgagtgtgagtgattccctgtagtattagtgttttttgttccatagtcgaaactacttgtgcccttagtaccataatcaaaactatcaatagcaatggtagcaccatgtgtatgaccaccaccagttaggtagatagaatgactgtgtgatggtaaattgttgctagataagctaacactactagagccaaagttactaccaactggccttgagttatcactgtagcctactaatgcccgaccttttgacaccaactcccaagtacctccacaaattaggtaggtagctgggttagcagagttcatcgaaagatgcaatgtaccaacagggtaagaagcctgaatagccttgtacaagttatttactgctcttgctgtagcatatttatctgcatcttcattataaaggttagatgttgtccagttctgaacattacttaaaccaacctgtgccttagttgtgttatgtgggttactcttatcattaatatgctgttgaacaagactattaacctcttctgaagacataatctgtagatttgctcttgcctcatcaacattagtaatgtctgataagttatttgcagcaactaactgaagagcattaataacattatctaaaccaatctgtgtcttagtaacaccatgagggttgtttcttaggcttgagtgtggagcaagtagttgctcaagagtgcatctcttatcctcaataccctgcttaagatggaagatatcactaaggtcaattggtaaggctgcttgaggcaaggcactaatttgaatttcacctactgccattattaagctcctacaaattcgtaagtataaagattttttgtacttgttgatgcagtacctgtttcagtcttaataagactccagccattagctactaaatctggttgctcagtggcaaaagaccttacttcaccaacagtaccatttgctttcttaaggaggtagtttaagatataattaaaccattggcgacccatcggctcaccccttaataagccagttgcctgaatttctggtggtggtaatacttttagctggttaccatcagcatctacttcatctgtagaccaatttaaaaatgccattaagaacttccttcttgctgtgatttatcttttctacctttgatgatttgagctacttcagccataacaccataatcaccacttgctacagtttctttaccaacgatgtagttgtctgtagcattataatttttattaacttttaagtaatctactgagccactattagccgttctgtcaatcttgaaataagcatctctgacaccagcatctactaagcttcctagcaagttcttctctaaagaacctcctgtattgttggtaacaagacctttgttagtatctgtaacgaaccagttatctttgtcatcaacaattgctaaagctgagtcagcaacctctacaggtgtccaagcagtaccatttaaagtcacatctcttaaaataaccgctgaaccaatagttgttgcagaaatttttgctaatgtgtatgctgtatcaagaacattgtttctcgtgttaaccctgattacaataccagcagtcataggtgtaatatgttcaaaaacctgtgaaaatcttgcattatacagtgtcataatagcattctgtaagaatgttggagtcgtatcagaacgtcttaagaaaatctggatatacaacattgccctatatgtttcatcgtcagcaccaagtggtctggggactttgattaaagtgccaatattatcaagttgctgcccaatcgccttcctaatattcctttcagtgtgcatctgccatgaaacatcttccaatgtctgcaactcatcagtaatagctttcagtaaacttgtatagatgaacttatctttaaactgtgttacagttctttcgtcaagagtcttatagtagacatcatcaattttctgaaacattattactccttagcaatggtgtactgactactttcccatacagtatattggtcaccatcaacagtaattcttgctgtagtgtactgtccatcattaggaggtactgactggttatttgaaagtgctaccttgatttcattaatctcaatacctttgatgacatcataaatatgtccgtagattctgttaggaataacatcactaccaactttcaaagttctgccgtaagcgttaataccttgaacaatactgtctctgatgtcttcttctgggattgttaggctttcttcatcatataaagagtaagatactttcacaaaagcatattttggtgttggtctgctgaaatagatattatgagctaagccacccaagtcgtaagctgtaccaaagatagcaccataagtcctaataccagcgggtttagtatcccagattgcttgagcaacattatcattttgaccaccaactacaacaatcttgaaagattttggtggaagtccttctgagcttgtctcttcagtatcattttcaacacctgaagcatctgatacaccctgaaccctcttaacagcagctacgattgcatcaagagttcctacaccagtaactgacagggattctaaatatctctgacgaagttctgtatcagtttcttcgtttctacctgttgtcaagtcatagcggttgtatacactgtcaagaccatctacagttgtttcaatctcgataagtgttccagctaatgcagggattgcaccaaattcctcagcaacaacgtcgtggattgtcgtaatttttgtgaatgtaaggaacgttgtggcagttacaaccatagggttggttcttgcaataatgtcaccttcatctttataaacccttaatgctgaaccatcattgacaacttctgctttcgctacgataccgccgttgattgcattagcaagctcagttaatagtactgtgattgaatctgaaggttttggctgataagagaaaataacgttatcaataataataacataatttgcatcagttcgtaaagagttaacttcaagaatagcttcaacacaatatgagggtgccaatgtgatgtttgaaactggataaaagatgttaccagcagtacttcttagtctggttgttgacggaatggttgcacctgttgttccagtaaactctacttgtcctcttgtagcctgagcaacatatctgtatacagcgtttaaagctgtaatatcatcgagattaaaaccttcagctttgtcaatcgttccaccatcatagatttctgacagaatttcatgggtgtctgctaaagaccttgcaattgaagctagaaatagacctagctgactatcctcagaaacgtcaaggtttggtgaaatatctctaagaagtcttgacttaatattgtcaaaaatttcctgatatcttagagtttgtaatcctgttgtagttaatcctgccattagatattaacctcttgcgtaatgtctgttaaaatatctgttgtagtagttgcatcaaaattaacagttacttttctctgagcattatccattgaagaagaatagttatagatgttagatacatctcttgtttcaaaaaggtatgctttcatgtaattatcaaagatagaagttctctgtttaaatttggcaagttgtaaatatgggaacccagcagatgtattaaagaagacttcaccagcccttaaaaggcatctgatatgaagtctttgagcaacctgagtagctttatcatcttctgggataattctaatttggttaccagtaatctttaaatcgccatgagccacatacactgaatctgaacctaaagtggcaacatagccaccacctagatttaatgcgaagtctgttttcattattgtgcctctgtagtatcagcctcaccagcagggtctgtccagtaataatggtgcgtgtgttcattaaagctcacaccagttgtatcactgataaaatctgaaccgtgtacttcttctgttacatacaagtttttagaaatgtgtacatcaccttcaaagtaaaagttaccatcatctgtaactcttaacacagagtcaccaaaatggagtctaactgctgttgggtctggtttaaaattctgtgttcttgtgcagatacctacgaaagctacacagtctgaaatatcgtgtgttcttctcatgtttgtttccatctgagtattcttgtcattgacaacgaagtcatctagaggcaacattgagaatgctaaccagcatctgtcattagtttttacagggaatgttaaagacgctccaccaccacttgggaattgaacaggtacaccagtaatctctggcattggcaacccattaatagagtaaagtggttttacagtggctgtttgagtctttgaatcaaaagactgaataatagctggtaaaccagtatacagttcttttctaaattcatcaaggcattctgaaacatacccagacattctagtaactggtgacttcattattccaccttttctaaatctagttcagttgtccaagcaccaccagtaaagtcaagattatgagaaagaccctttactcgatactgaccttcaaagtcttcactttccctaatagtgacgttatcacccatcttgattctcccatctaaatggattttgcaacgaactccggtcttaactttaataactgtcttattctcttttttaagaacctttctagttcttctgtagtaaccttgcaaagaatcaataacgttatatgggtaaatttcccaagaaagttgtctggccttagcattaaaagggactactcggatttgcttgttaaatgtataccaacgtaggctactctcttcacaaacctttgtaagtgcctctgcaacactcccccaaacactaaaaccattcttgtaagtataaccatcaatgcttgaaaggtcttcatcaataagagagaaacctaacctgtttaccaaatctttaattacacttttacgtgttgtccctgctttataagagattgatgtcttaatcgtggttctttccattttatcattggagcagataacctttgtaatcatatcaacaccacgcttatatgtataagcatactcaatagtgcctagatagattaatggtagattatcatattcaataataaggtcgccgtttgcatcccttttaaaaccagtagtataaccagctctgagcataactgttgcaccaactgttttaaacttggctctcatctctttattaaggttgtagatttcaaaagtggtatcatcagaggttactttattcttctgagacgtataagacacgttacaagtgaattgtaagttgtcgaaatagtccatttgcatagagtctttagcatggcttgtaggtttatcattaaaggctgtagtttcactacctacagccaattgatagcacctaaaagaagccccagcagtgctatcttttacagacattataagttctccattaatctcatatcttcttgagtgtaataattaagctcaaatgccttttctcttccgaagttatttctggtaggttgtaaatcagtaccgtacattcgttcaacaaaaagctctccagctaatgaaggaattacatagcgtcctgtgattgattggtctgcaaggcatttcttttcatgtaataatacattaccatcaacatcagatagcgtcaagaaccatctgtcaagcctttctttatactttaactcaattacaaagacagtaccatccagagttacagtttgtgtagaccattctgtatcaggaacaggaatatattgtgacattaataagtctccttcttattcggattcactgagtgcttttgtaacgcatttccagttccagcagttctgtttaatgttgacctagcatttctttcagcctcttcactaaatgtggtaacaccctttcttgtctttgcagacattgaacattttccaagagccttgtcttcatcactagtaagttcccttacgccattggcatttaggtcgaataacaactgacagtttaatttgccattacctaaacttgttgtagtattccctgtattcttcttagaagttgcaccaccatcattagtagtagcggtttttcctgtcgcagccgaaatatcagtttgtcccacaacagctttgaagttaatttcctgaaaagttagctggattcttaggccgttagaaattccaacatctttagaggctttaaagcttgtaataatggaatcgtcaattttaattccatctttacagatgactgaaataatctgcttctggtcacgccagttttccagagtgtcgatgaagctttctactaattgaccctgacgagttaataataagctcccttcatagccaactacaacgacaccactgatagtgattgttctgggtgctctttgaacattatctgtaacagtttgccctgattgcatgttctgtgtagttacctgcatagggctgtcaaattccatgttttcagttgctgataaggttaagaaggcatctacattatctcttaagtggaaatagatgccatctttgccactatatttgatttgcatattagaatcccataacattattcttcctctggatagcttgaacttagaagaatgtcttcttgattcttgtcagtaatatctaccatcttagtagcaatttgtttaccatcaagcttgaaagtaacattcagggtttgtttagtctgcataggtaaaccagaaggtgtcatcatcatcggtgtctggttgaacttattggtaaaattatcaagagatgttgataacttctccatgagaatatcccaattagatagaccattatcaattaacttgatgttaccatccatatcttgagtgtactgtgcaaactgaagttgcccattctcatcaaagaacattggtctttttggattcgtaatatttgcaacagcattttcaaacggttttggtaatgatacgtcactgtagtttttagcagcatttgggtcagaagacctagatagtgcaagcgttgctgctgcaacaccgactggagatgcaagtaatggtgcagcccgtggtgctgctttttctgcaacctcaataccaatcctctgaaggattttcctcacaattgctgtatcaaagaacccagtgataagctttgccattttagctatcgttgctacagcaccaccaatcatagtgactgttcccagaaattcaccagcacttttgactagtttttgctgactactgtcaaggtctttataccaagctcttgcatagtagtatagtgcagagactctgtacatgaagtctgtaacgaagtcaagtaggttaccagcaccttttaaaagattcccaatcacaagacctaaagcctctgtagtacccatagaaccttgcaagaacattgcaacggagttagataactgtgaaataccctcactagagccgttaaatagtgctacaagtgtgttatcccacatagccttagcttgacccattgatgtagcggtctgcttagacacggcattcataccacctgcttgcttgaccagttcagccattttttcagatacttttggcagaacgtcttgagcaaggagtttaccgtctttcatcagcttatcaagttcttgtggagttttgccaatggcatcagcgaataactgcacagcacctgctaaacggtcacctaactgtccacggagttcttcagcttgaattttaccttttgatgccatctgctggaatgcgaccatgatacctttcaagtcttcatcagtagcacccctgatacgggcaaacattacagcattcttatagaactcttgagtaccctgaaaaccaagtgttggttgagcaccagcagcaaagtttgaatactgcttcatagtatctgtgtagttctgaccaatctggtgtgagaatgatgcagcgaacattctggcttgctgtgtatctgctccaaagatagctgtagaggctaactgtgcagattgtctttttacacccgcctcaatagtcttttgggatagttccagtaaagcataagctgaaacaaaaccaccaactaattgacgtaatgatgcgttagctctatcctgtagccaagctgcttctttaactgattttagtctagcattttctgcaataacccaacgtttggttacgtcgatgagttttttaacttccatctcatactcaccaaccttaccagtgcccttgtatttattataaatactttgtaagcttcctctgaaagaagctgccatctggttaccttgaccaccaattgtttccagtctacgggttaaccctgaatagaagttgttgttaaacattctttccatttgtctctgagcaacatctactctcgggcctctggatgctccaccaccaccaacagggggatttccctttccacctcttcctctacctgtcttaatagtgattttaccgtcaaccttcatagcatctcttaaggacttgttaataccttttgcagtcttttttgcttgagtttcaagtctcttaagagatttaacaccttgcgaatcaaggttcaaggaactgttgagtgctttattgattctgcccgaagcagactgagcattttttacaattctattaagtgcttcctgagaacttttattaggcttcacatcaaaggctttattcatgtttctctcaatacgttgagcagcttgcatagacatcttctcaactctctgcaagcctttaattaccttttcactgaaaccaagttccacaatgaagctatcaactgtatattgtgccattacatttttcctgctcttctaagttcgttgtaagcaatttcctctttatacgacctctgaatttcaagaaattgtctcaatgataataaatcagagaatgtcatagcaaagagttggtcaagtgtttctttacacccttccataccataaatagcaagcacaaatttcatctcgtctgcttcttcataggttgcctctacagcagcatcagtcagtggtgtctgtagagtgttacccatgtttactgagaagttaggcttttgaaaatgcttgcttcgaaaaaacttccgaagtttgcctccagcgcaaatgctaagtaatcaataaattcaccgtagtttgcttggaagtatgtatcaatattaagtgggaagtcatcaacagttgccccttgaaatagcagggtagccatttcttctaggttaatttcctcaattctgtcaaaacaagcttcaacaagttctttaaatggaaccattggagcttctttcttatctttatccgtcagacttgatagcatctgtgcaaatgttggaacaacaattttacccaacttcatagacatcttaataccgtctcttgccccaagcagaatgatatttactttcttaccattaattactctagattctgttttcattgtgattccttaatacttttaaaagaaacaaaaaaggggaagaccttttaaagtctcccccttataggatttattaaacacttgacgctggaattgtagaagtgtagtctagcttctcacaaccaaaaatccaagttttagagttctggtcacgaccaagttcaatctgtggtaattcctgcaaccaagcattaataccagttgccagaacagagcctgatgggtcgtagattacgaagttagaagagatatcttcttcaagttccatattgtcttgtttagcttgaattgcagaaagcatctggttagagagagaagtctgcattaactcaatctcaatagtacctgtcttgtctgcatttcttgtcagagcaacctgaccacctgcacctacaactggtgtaataagtggtgatgttctctgtagacgtaagaatgagtctggggcaaagccttcaatagcaatgccgttccagctacatacaacgtctttaggggaatattgctgatacatagccattccaatttacctctattattcgtaagccactgtacctttcaagtcaacatccaagatagcacctgctaagatacctgcgaaggtaacatctttcaggatacgagctttcttgtctgccaaagcaacttgagaggctttaggaacattaactgtgtaagatgacaggaagtttctgttaactgctctttgtagagaggtttcaatgacttgacgaatacgggtaataccagtatcatcataagtaatcttaccacccttctgattaattagcaagtctctcagagaagttttcaggtctgattctaaccagtcaacaccacggatgatgtcaatccattccccaccagaagtaatccctctacgaaccactggaacaccaccatcaaggtcaataaagttacagtgacgtgtatctaaagcagacttctgaatacttgtcagaggtctctgattagatggctgtagagaagcagctacaccagttaactgtgcattaccccaagcaattgaacctgcatcatatggagcaccataagcaatgtatgccatctctggataatcttctgctgctgtgtgatgccacaaacaaactgtacgagtgtacatattcttagcaagctgtgctgggacatcatttgcactagccaattctgtgccttgtagtgctgctacatcagagttagccgtgaagaaaactttcttacgagcctgaatctcggaagccattgctaagacaaactgttgagttctatcttctgctgcaatgaaataccagtcggtagagtaagtttcgatggctgccagagcagttgatgctgtatcagcagttgtacttgcaacatatacagtctgtgctgtagttgttactttcacaaagtcattatcaccagctttggtaataatcattgtggcagaaccattgctaccagttacgttcacagaaaccttatctttgattgttgggtcagcttcaatctgtgttttaaactgttgcaacacgttttcagcagtgtcttcagcttgtgctgtatactgatatggttgagaaattccaccgccagcagctacagtaattgagtagtctgtactttcagtaacggcattaggaattgatacagtgtactgcatagcacgtctaccaatataaagctgagtcactttaggagtctgactccaaagttgtttagcagccttatatgcagcagtattctcatcgaaatcttcagcaacttcagttaaggaagtgtaaccacgaactctttcttcaaaattatctgttgaagctaagaatagtggtaagccaaaaccttctcttgtagttcctgcggtgttcaatgtaatatctacattaacaattggattccacatttattttacccctttggagtctacatctagatggatagtatattctggtggctcttgtccttcttcataaactaactcgccatcgacaatgacacgctcaataacacttccacgttcatctttcaggactgagttttttacaagagttacaacaagaggtgcagaattttcgaaatctgtattgagataagtataatcatttgggattgctccagtgtctagtactgtagctcctgtttcttcaaggattaaatctctgactgaactcatctctaatcgttgtttaagctcaagcataatactgtgagcacctttaccatttactgtaatcaatacaggaatctgaaaagcaattctgtaacaaactacatcatcctcaacaaacttatcaagaacccaaccataaggtgttgcagcatcttgacagtatacggtgataaatggttggtcaggttttaaacctttgtcatttgagttatcagaagggtaagctctaattacgtttggcctattatttttatcacgagctagtctgtgaccaataacatccactaaggttctaactagacctttttcaagttctgctgtttctaactgcattcattttatcccttctaataatgatatattcgtaatgggacgtatgggctaattgttgtgaccaatccatagtcataaatacttcatactcatgaccatcaatcataacaatatcagactcattccactctacatcatctgaagttctaagtttatatgtagtatacaggattcttgtatcggtgagtctaataccttccggtaaagcaatctgtgtaccgttctttacagaacctttgatgtatggctgaatattacctttgcagttaacctcaacaatatcttgtgaagatacccaatcaccatcatcattataataaccgtcttcagagactttacgctttactacaaagctgtgtctgtttaagagtctcattttttaatacccttcttagtagaaattttataagcaaggttatcccttaagtcacctgtctcaacaagaggtgcattaaaaccttttttcttgactgtggaaggtgcgttaggagggaggatagcagaattaccaaaacctcttttaattgccttctgagcattctttgcaaatgcttctaaggtatttgaagggtctgtgttgagactgctaagttgcttatatagattcttcttagtctgctctaacaaggtctgtttgtttagcatcatagtgatttcaaacaacctacgatatactttaccagaagctgagggaaccccaataacttcttgtaaatacattaaagcaggataagaaaaaccagagctatgttgaccttgttcttgaaaatacccaacttgagcattagccgtttgcaagttcttcatagccccgactaattttgctctagcggggtgaataacccttttaaccattattcatctcgctcgataataaatacgccattaacacggttgcagtaatctctgccttcgtatctggcagcatcaccatattctgtgtatttcttgacagagcaaggattctgacgacgcatatcaatgtcgcactgattaataccacctgcatagggtagtcctgaaacagagcttttaacaaggtcatcataaacagctttcagagatttgaatctggaagagttacgtaaataaacaccaccaaccttctcatctcccatcttggctacttgaaagagtaaatatttaagagctttgatggctgctttcttttcatcctttccagattctagatagaaccactctagcacagactgctcaataagaatttcatcattattagtgtctgtgcagaggattcttactctatcaagagggttattggctgggtcgcctgtataacacattcataaccctcctaagtttattagccacctttagcgtcagcgcgaacatctaccagcaactgaggacgagtgcagtatggcagcatgtaagagtgagcttcgaagtcaataccttcgtcacggtctttttcgtattcgaatacgtacagttcctgaccaagtgtatttgcataacccatcttaggacatgggccatatgccacttcgaagatgttgttagcttcgcccagcatagatacgttagggaaggcatgaccaacaccaacagttgctgctacactatcaatgctcaccagagtgtgaaccttaccacgcttgtctttgaacttaccgttgtactggacaaacttaacaccaccgtagtagaaggtgttcatatgagcctgaacgccgtcagtaccaccagttctcagagaaccagtaatctgttgccaagccagtggagtctgctgtgcaagataagcgtcacgaatcttaggatgcttaaccagtttgctgaagaatacacggtcaacaactacgtgaatttcttcaccgttgattacagtgccagtcttagcttcgtcttccatgtgcatgcgcagttcttcaatagaagcgtcgatgtcagcattaggattgtcaaggtcgaagtaaacagtcttcttctcaacgtcgaactgcttgtacaggtcagcgtacagagtaccacgagcatcaacaactttacccttcagagcttgcataaacaggaactcacgagtaatatcgaacttggtacgaatcttcatcagcttcttagcacgtactacagcttcagtagtcagttcatttgcagtgcctggctgacgtacaccctgaatctcatcaggagtaatgctttcaacttctttgaagtacatcattgggaagctgatttgacgaacacgctcaggtgcgctagtctctgctttacggctatcacggtctaccgcatcaagcaagctaacatcccaatcagtcaagtccataaggaaagtagtttgggtgattggtgctgaacggaacagacctaagttggaaatatacccataagtatttgggatagactggacttcaccagtcaggtcagcaaggaaaaatctgcttttttcagaattagtcaacattgtaaaattctccagaatgtcttattattgttattacaggccagttggtacgaaatcaatacctttagcagccagagctgccttaacactatcagcattaacacttgcttcaagagtaagcatgtctttcagttctgcatcgcggtagatgcctaccactttcaactgaccatgataagacagttgtgagtctgcgtagaagttaacgatacatacagagtcagcctgagcttcttcccctgctgcaactttagtaccgtctgctttcagaacttcacctacacggtattctgttgaagcaactggagtgtactctttacgagagtgacctgttggggtaacctgttcccaaagaatgatatcattcagaggttctcttttacctaacttagtaaaaccttgatatgccattattgtgttccttatttgataagagattttagagcattctggagagccagtttgcgttgttcagcggtgtcttcggaagcattcttagctggttctttttcttcttcaaccaaatcagcttcgccgtcattacccatttcttccatagcgttggaatgttcaagaacagcaccagcagtttctttcagcttggtaatttctgattctttttcttccattgcagaagcatgggatgcgatagtttcattaagtttctggttagcaccttccatagcattcataaacagaacactcagagggttatcaagaccagcacctaaaatagtcgttgcagcttcttttgcatcaaacccaaaagcctcagcagaagcagaaatcttattagtcaaatctgacaaagcagcttcctgttctttagctttcatttgagcaacctgaagacgcaaagcttctagttcttgcttttcttgttcagtcatttcttcacctgaattgttaacgtttaaacttacaggagcctcttcagaaccttgtaagtaatttaagaaatcatcttgagacatgattgagttaattaaaccaagttcaagagcttcctgagcagaataaacattcgcctcagtattctttacagcttcttcagagagattacgagattcagctacaaaacctgtgaaggttgcgtaggtatcattaattctcttttgaagtctttctttgctttcttctgaaagtgcttggaatggtgaacccatacctttaaactcaccagctttgatgacgttaattgttacgccattcttttcaaatgccttagttaattcctgatgaaccataattacaccaatagaaccaacatctgcatctggcgatgcaataatttcttcagcagaagatgcaagagcatatgcagcggaacaagcgaactcatctacataagcaataattttcttttggcctcttgaagccataatgtgacgtgctaattcaaagcaacctgaagcttcaccaccaccagaatcaatgtgcagaacaatagtcttgattgactcatctgctaaagcttcgtcaaagcctctgcgtaaaccttcataagagcttaaaccacctgtacacattgcatcaatgaatgtcatacgatgagttaaaccacccataataggaataatagcaatgtcatcttttacttttaaaagacttcttgcttcacctttgggtttatcaaagtttactgctgcttggacatcacccagcaatctgttattcacataagttgctgctgagtgagctaatgattcagtggctagtaaaggttggttgaacaatctatcagcaagtctgaagatattcgaactcatttttactcaccctatttgtttaaatctacagagactgaagagataacatagataccttcttcagcgaagaattgaggtttgctaagagcaccagttgccacgcatttatcgttagcatcccacaagctatagtgtgagaccgttgcagaagctgggacagtaatattaactgtgtcttctgaggcaatcaagccattatccggttcagaaaaataaatagccactggctgagtaaccttatttgctgtagggtctgccgttgggtctacattgtgtaaaataatagtcgttggggttagcgtggcgaggattttattcttaccatcaatagttaatgttcccattaattaaaccttactttttgtttaaggactgttgaatggttaccagattcatctaccacatttacaatcatatcatatactttacctttgacaagtactttgtaatcattttgtgaaaaaatgtattcaagtctgtttgtttctttattaactgtcattggagattgaaatgcagtgtcgagggtaatgaaagcggcttcaatgctattaacattgatacgtttattctcgcagttatataactttacacccaaaaggcatgaactgtcaaatggaattttaacaatttcactacaatttcctgaaataaacggttttccactcattggtgcatcaagtaacctacaaatagtgaaaacgtcagagattccaccatcactaacataacctgaaaggctgacccttgagccagcctctacaggtaacttatcagtaatgataaggacacctctatacgaatgcactcgtgtagcatttgaaatagagataacttcagccattatttatttgccttatttgctgtgcttgggtctttcgctgaaggtgtctttgcagtaccttctccagcggtcttataaccatctcctgaacggctttggctattcggagaaagcttttcagatactggctgagactcatcagcaggaggaagaccaatatgctctctaagtttgttagatagctctttgtcaacttccaaagcacctactgcaacagtcttctgaatataagagccaattgcttcaaggtctggagtttcgatatcatcatatgtgatttgtacatgttcttcatcatcccacatattaagagcataagtctgtgcaactaaatcacggttaattacgttcttaatttgcttcagcagaatatctactgacattgctaataagcttgtcttagaatctgcaagagagaatgaaccatattttgactgacccatagcaagaacatctgacataaatgccatcataatctgcttagaatatctgtcaataatagaacctgtatcatatgctttagcaccctgtctagaaactaatgagaactcaaaaatatcctctttagtatctgggtcaatatatctaggccagattaaacctgctctgtcgttagcaatcatatcattaacaacagttttgcagtattgtacgaaagctttcttttcaggttctgcattttcatccagataatctggtggtaaaccaatctttggcatacctactaagtctcttgaaacaccaacagcttcatactcttcaatctgtactttatacttccacggtacataagcattaagcaatggtgaacgaccttctgggttaccatactcatcatcatacttaaacagcatgaatttagctcgtgggagttttcttgttagtggtctttctccaagattaattgctccagcaatatgtgaaacatttcttagattttgtctgacaccagtaacttttctaaagtcttcgtcaaaataccacttatcaagtgttgactggtttctgattggtaatttagcccacccaattagaccatcatcaaattttgactggtactttccttttttaccctgacgtttcttataaaccttttcgttaacacagaacccataagtgcagaatgacattacagagttaataaaatctgcccaatcatgctccatgtcatccattaaagaattaaagaagtctgctctttcaagcattttagggtcttgctcttttccctttggaggtacgaatctccagttgacttttctgacaaacatcttaataatatttacagatgctgctacagcagggtcacgcatcattaattggaaagttttaatactttcagggaaccttagtgcctgacgaggttcttcatagattctaccattcttaaccttcagacccaaagaacctacttcacccattctaaatggtggtaagctttcttgtgtttctgtaatttctgccattcttctcacctagctatcaacgtctaagcccctcatatgggtttcctctcactaagtctgtgtgagcacccattgatggtggcttgaataatttaacttcgttaagactgttgaaagcatcactggtagcatccacttggtcatctttagttttaccatcaccacaaaagccttcaagttcttgaaagtaagcttcgttccaactgcctctcaagacttttacaagtccagcttcagaagcagcagaaaatcccgcaaagcgggtaactttatctttatttgttggtttagctcttgcgcgataacctttctcggcaagtttcctgatgagggatgttgcataggatttaccagcagcgcctgggtcttgagggataaaaataccagttcgcttaccgtcactttcagcagtcaaattaatttgtgtttcgactccagagggtctatctctaaatcttactacatcaatgatataatagcaaccgtcttttttagatttacccaccttaacacccgctgtccagtctgggttagggttaatttcagatggtaaagttgctgctaagtcccatgctctgacatcaaacacatcctctgggagtgaatcaacaatttcacaccattgtctttgccaatagtttgaaccttctgcacgagccttccagttaccaaaacgaagtcttgcaacgtttacaggtgtgttgttttccaacttaccacgatatttaggttctaagaagtcaagaattgggttatcatcaatcgtaccagagatgaaggtgtatgtctgaggaatctcaagagggaacatttcaagaatcttgtctctctcccaatcagagaccatcacaccatcattcataacataccaacgaatacgaccacacttctcagggtctggatatccttcttcatctaagaatggttctacccaatcataaataaaatggtctctgtctgggttcatagaaatcttcatgtatgaatcaccttcagccccagaacgtagacgagtctgtaggtatgaaatctgtgaagcagagaagtgtgtaccttcgtcaaagtaaatagctgagtactcaataccctgatgaccttcagcgtgcttttcaagttctaggtaggtaaacttgatagttgccccagaagggaacgtgatagtcatcttctgctcgtgaggaaccccaccaaacttaccaaatagtttctttgcagcaggccataaaccaccttgtaactgtgttgtatttcgacggaaatatacagcgttatagttagggtcttcaataaaccttaaagagtccattaacaatgcagcagtcttaccagcaccagcagcaccaccatatagaaccaagtcagcattagtatttaaaaagacctcttgagaacccggctgaggggctacatagttcttatcagtcatcaatttgaagataagtctaacttggtctggtgtgtatcttaataaagtcagaatttgagttggaaggaatttagaggggtctttaccgaatgatttgataatttcttttacttcatcagaaagccccaactctccagctaggactttcctaacatcttccactcgcttctgcttaacagcatttaagtccattaagcacctccgcaaatagaattattcagagtctgtagcagtctcttctttcttaacttcgaactttgcatcaacagcatcaagaagtgcatccatagatgcttctttgatatcaatacctgttgctacagacaatctgtctgcaattgccatgatagtattctgcataaattctagcttcttattagcttcataaagttctttataaacggtctcgctcattatgttttctccaaataattaaccatgtttccccttaaaaaagacggtacagagaccgtcaaggagaaaccacaatgtacgtcagagacatactgtataaggcttcttataaagtataactctgtaaaaaagccctgtctaaaaatactcttcggtagcgaaaggaagaatacttatagcagggcattattattattttaattgagagagagtaaaataacttggagaatccgaagggactcgaacccttataaacctgttttgcagacaggcacatagccatttctgtcacggattcaaattggaggaagataccagacttgaact